(19) United States
(12) Patent Application Publication
(10) Pub. No.: US 2025/0152536 A1
(43) Pub. Date: May 15, 2025

(54) DELIVERY OF AN ANTIOXIDANT COMPOUND FOR TREATING SEIZURES, VIRAL INFECTIONS, EXPOSURE TO CHEMICAL WARFARE AGENTS, AND OTHER CONDITIONS

(71) Applicant: Beyond Barriers Therapeutics, Inc., Glencoe, IL (US)

(72) Inventors: Ryan Kole, Glencoe, IL (US); Brian Reinhardt, Glencoe, IL (US); Jeffery Lewine, Glencoe, IL (US)

(21) Appl. No.: 18/838,178

(22) PCT Filed: Feb. 16, 2023
(86) PCT No.: PCT/US2023/013219

§ 371 Date: Aug. 13, 2024

Related U.S. Application Data
(60) Provisional application No. 63/310,986, filed on Feb. 16, 2022.

(51) Int. Cl. A61K 31/198; A61K 9/00; A61K 45/06

(52) U.S. Cl. CPC A61K 31/198; A61K 9/0043; A61K 45/06

(57) ABSTRACT

Methods, formulations, and kits comprising an antioxidant compound for treatment of seizures, viral infections, encephalitis, exposure to chemical warfare agents, and/or exposure to a pesticide. An antioxidant such as N-acetyl cysteine (NAC) or a pharmaceutically acceptable salt is delivered by administration to a subject in need of treatment. Methods, formulations and kits may also comprise a cholinergic muscarinic antagonist, a cholinesterase reactivator, or both, and/or an inhibitor of a purinergic receptor, and/or a benzodiazepam. Kits for treating the subject comprising (a) at least one antioxidant compound or a pharmaceutically acceptable salt thereof, (b) an intranasal delivery device, and (c) an intramuscular delivery device. The pharmaceutical formulations can be pre-formulated into vials or cartridges that can be loaded into the intranasal delivery device or intramuscular delivery device.

### CROSS-REFERENCE TO RELATED APPLICATIONS

This application claims priority to and benefit of U.S. Provisional Patent Application No. 63/310,986, filed on Feb. 16, 2022, the contents of which are incorporated herein by reference in their entirety.

### FIELD OF THE INVENTION

The present disclosure is directed to formulations, methods, and kits for treatment of seizures, viral infections, encephalitis, exposure to chemical warfare agents, and/or exposure to a pesticide.

### BACKGROUND

Organophosphates (OP) act as potent and irreversible inhibitors of acetylcholinesterase (AChE) in the peripheral and central nervous systems, with OP exposure resulting in a hyper-cholinergic crisis that can lead to respiratory distress, cardiovascular dysfunction, coma, and death. OP exposure can also result in an imbalance in Glutaminergic and GABA mechanisms leading to seizures, status epilepticus, and excitotoxicity plus trigger secondary injury involving increased oxidative stress and neuroinflammation that can persist for months after the acute seizure period. At both lethal and sub-lethal dosages, seizures with progression to status epilepticus (SE) are also common. Animal models of OP poisoning demonstrate survivors to show neurodegenerative changes that increase with time (see review by McDonough and Shih, 1997; Joosen et al., 2009). Changes in EEG patterns, neurological changes and changes in behavioral tests are also observed post exposure (McDonough et al., 1998). Studies of victims from the terrorist attacks in Japan (Miyaki et al., 2005; Nishiwaki et al., 2001; Yanagisawa et al., 2006), as well as data from cases of organophosphate pesticide exposure in agricultural workers (Steenland et al., 1994), show that humans also exhibit neurological and behavioral changes post exposure. Organophosphate (OP) poisoning can have short- and long-term neurobiological consequences, even with administration of the current standard-of-care countermeasures. The current United States Department of Defense standard of care for nerve agent exposure is to treat with the Meridian Medical Technologies Antidote Treatment Nerve Agent Autoinjector [atropine (2 mg/0.7 cc) and pralidoxime (2-PAM, 600 mg/2 cc)] up to three times, then, as needed, followed by one Meridian Medical Technologies diazepam (5 mg/2 cc) autoinjector or one midazolam autoinjector (currently under development).

Much of the brain damage in both humans and animals appears to be caused by overstimulation of excitatory amino acid neurotransmission in relationship to seizure activity. At moderate-high exposure levels, animal studies clearly indicate that countermeasures aimed solely at the hyper-cholinergic crisis, such as atropine and 2-PAM, can advert death, but they generally fail to completely stop the development of seizures and subsequent neurobiological problems (Shih and McDonough 1997; Raveh et al., 2003; Weissman and Raveh, 2008). In rodent models, these behavioral and histological abnormalities may be mostly (but not completely) avoided if additional GABAergic agents like diazepam or midazolam are given within the first five minutes of seizure onset. However, a delay of even only 30 minutes markedly reduces protection (Gilat et al., 2005). Converging data indicate that the duration of active seizures is the critical predictor of the extent of subsequent neurobiological compromise. Countermeasures that stop the development or seizures or reduce total duration to only a few minutes mostly, but not completely, preclude the development of long-term neuronal damage. In contrast, if seizures progress to SE for even only a short time, long term damage may be severe. Animal and human studies demonstrate that SE leads to neuronal damage through both primary mechanisms related to excitatory amino acid toxicity and secondary mechanisms related to increased neuroinflammation and oxidative stress. Importantly, once triggered, these secondary mechanisms take on a “life of their own” where stopping of active seizures does not quell secondary injury mechanisms.

Substantial previous work has shown that oxidative stress, resulting from high levels of reactive oxygen and reactive nitrogen species (ROS/RNS), plays a key role in pathology of status epilepticus that develops during the period of secondary injury. Following seizures there is an increase in oxidative stress and a reduction in levels of Glutathione (GSH), the brain's primary antioxidant. Even with countermeasures, some functional and structural impairments persist (data from Lewine et al., 2018).

Studies in rodents have shown that inflammation and oxidative stress may themselves be contributors to the generation of seizures, ictogenesis, in animal models of acquired epilepsy (Vezzani and Ravizza, 2019). For instance, the pro-inflammatory cytokine IL-1β is upregulated during seizures and exerts proconvulsant effects via multiple pathways, one consequence of which is potentiation of excitatory responses mediated by the N-methyl D-Aspartate (NMDA) glutamate receptor subtype (Vezzani, 1999)(Viviani, 2003)(Balosso, 2008). Additionally the bacterial endotoxin lipopolysaccharide, which induces a robust inflammatory response, lowers seizure susceptibility in rodents (Sayyah, 2003)(Galic, 2008). One example of a specific link between oxidative stress and ictogenesis is the high-mobility group box-1 (HMGB1) protein, a chromatin component released by necrotic and apoptotic cells (Scaffidi, 2002)(Muller, 2004). HMGB1 is redox sensitive and its oxidized form has been shown to also potentiate NMDA responses in hippocampal neurons, and effect that was not seen with a mutant form unable to undergo oxidation (Balosso, 2014). Antagonists of HMGB1 reduce the generation and severity of seizures in kainic acid and bicuculline rodent models (Maroso, 2010). Additionally, administration of NAC and sulforaphane, which increase glutathione via complementary mechanisms, reduced oxidative stress and seizure incidence in a rat model of acquired epilepsy induced by electrical status epilepticus (Pauletti, 2019). Therefore, IN-NAC may be beneficial not only in ameliorating sequelae of OP exposure, but may also the reduce inflammation and associated seizure pathology from other chemical and biological weapons agents. For instance, IN-NAC may reduce inflammation and seizure pathology seen in severe cases following neurotropic viral infections.

Neurotropic viruses can induce neuronal injury, modify neural networks associated to seizure cascade, and produce proinflammatory cytokines (King et al 2008, Singh 2011, Vezzani et al 2016, Chen et al 2020). Most importantly, a previous study has shown that the number of inflammatory cells correlates to post-infectious seizures during CNS viral infection (DePaula-Silva et al 2018). Neurotropic alphaviruses such as Venezuelan, Western, and Eastern equine encephalitis viruses (VEEV, WEEV, and EEEV, respectively) are known weaponized biological agents (Ronca, 2016). Other important neurotropic viruses include Dengue fever virus, Yellow fever virus, Japanese encephalitis virus, West Nile virus and Zika virus of the Flaviviridae family (Volz et al 2016). These Flaviviridae viruses are also associated with oxidative stress induced neuronal death. (Zhang et al 2019). Additional viruses this application addresses are Filoviruses (Filoviridae) including the Marburg virus and Ebola virus, Bunyavirales including Rift Valley fever virus, Crimean-Congo hemorrhagic fever virus and Hantavirus, plus Arenaviruses (Arenaviridae) including Chapare, Lassa, Machupo and Lujo.

Supportive measures are generally indicated for acute lung injury secondary to exposure or mustard gas and other chemical warfare agents. Patients may require supplemental oxygen, intubation and mechanical ventilation.

### SUMMARY OF THE INVENTION

In an embodiment, the present disclosure provides a method of treating seizures or neurotropic viral infections. The method comprises intranasally and/or intramuscularly administering to a subject an effective amount of at least one antioxidant compound or a pharmaceutically acceptable salt thereof.

In another embodiment, the present disclosure provides a method of treating seizures following an exposure to an organophosphate OP, such as a chemical warfare nerve agents or OP pesticides. The method comprises co-administering to a subject in need of treatment for a seizure an effective amount of at least one pharmaceutical compound (for example, an antioxidant compound or a pharmaceutically acceptable salt thereof) and 1-O-n-Dodecyl-β-D_Maltopyranoside (DDM).

In another embodiment, the present disclosure provides a method of prophylactic treatment of seizures prior to an exposure to an OP, such as a chemical warfare agent or OP insecticide. The method comprises co-administering to a subject at risk of exposure to a chemical warfare agent or OP insecticide an effective amount of at least one pharmaceutical compound (for example, an antioxidant compound or a pharmaceutically acceptable salt thereof) and 1-O-n-Dodecyl-β-D_Maltopyranoside (DDM).

In another embodiment, the present disclosure provides a method of treating a generalized seizure. The method comprises co-administering to a subject in need of treatment for a generalized seizure an effective amount of an antioxidant compound or a pharmaceutically acceptable salt thereof and 1-O-n-Dodecyl-β-D_Maltopyranoside (DDM).

In another embodiment, the present disclosure provides a method of treating a focal seizure. The method comprises co-administering to a subject in need of treatment for a focal seizure an effective amount of an antioxidant compound or a pharmaceutically acceptable salt thereof and 1-O-n-Dodecyl-β-D_Maltopyranoside (DDM).

In another embodiment, the present disclosure provides a method of treating exposure to a chemical warfare agent and/or exposure to a pesticide. The method comprising intranasally administering to a human subject in need of treatment for exposure to a chemical warfare agent and/or exposure to a pesticide, an effective amount of at least one antioxidant compound or a pharmaceutically acceptable salt thereof. In some embodiments, the subject has a protective facial covering (such as a gas mask), and the method comprises intramuscularly administering the at least one antioxidant compound or salt thereof without removing the protective facial covering. In some embodiments, the method further comprises detecting presence or level of the chemical warfare agent and/or the pesticide in an environment around the subject. If the chemical warfare agent and/or the pesticide is not detected or is below a threshold (for example, a level recognized as safe for humans), the method can include removing the protective facial covering; and intranasally administering the at least one antioxidant compound or salt thereof to the subject. If the chemical warfare agent and/or the pesticide is detected at a level above the threshold, the method can include refraining from removing the protective facial covering; and intramuscularly administering a second dose of the at least one antioxidant compound or salt thereof.

In another embodiment, the present disclosure provides a pharmaceutical formulation for treating a seizure, a neurotropic viral infection or exposure to a chemical warfare agent. The pharmaceutical formulation comprises at least one antioxidant compound or a pharmaceutically acceptable salt thereof and 1-O-n-Dodecyl-β-D_Maltopyranoside (DDM).

In another embodiment, the present disclosure provides a pharmaceutical formulation for treating an alphavirus infection. The pharmaceutical formulation comprises at least one antioxidant compound or a pharmaceutically acceptable salt thereof and 1-O-n-Dodecyl-β-D_Maltopyranoside (DDM).

In another embodiment, the present disclosure provides a pharmaceutical formulation for treating a seizure. The pharmaceutical formulation comprises at least one antioxidant compound or a pharmaceutically acceptable salt thereof and 1-O-n-Dodecyl-β-D_Maltopyranoside (DDM).

In another embodiment, the present disclosure provides a pharmaceutical formulation for treating a seizure due to epilepsy. The pharmaceutical formulation comprises at least one antioxidant compound or a pharmaceutically acceptable salt thereof and 1-O-n-Dodecyl-β-D_Maltopyranoside (DDM).

In another embodiment, the present disclosure provides a pharmaceutical formulation for treating a subject diagnosed with encephalitis. The pharmaceutical formulation comprises at least one antioxidant compound or a pharmaceutically acceptable salt thereof and 1-O-n-Dodecyl-β-D_Maltopyranoside (DDM).

In another embodiment, the present disclosure provides a pharmaceutical formulation for treating a subject following exposure to weaponized biological agents. The pharmaceutical formulation comprises at least one antioxidant compound or a pharmaceutically acceptable salt thereof and 1-O-n-Dodecyl-β-D_Maltopyranoside (DDM).

In another embodiment, the present disclosure provides a pharmaceutical formulation for treating a subject infected with a virus selected from the group consisting of Dengue fever virus, Yellow fever virus, Japanese encephalitis virus, West Nile virus and Zika virus of the Flaviviridae family. The pharmaceutical formulation comprises at least one antioxidant compound or a pharmaceutically acceptable salt thereof and 1-O-n-Dodecyl-β-D_Maltopyranoside (DDM).

In another embodiment, the present disclosure provides a pharmaceutical formulation for treating a subject infected with a Filovirus (Filoviridae) such as the Marburg virus or the Ebola virus. The pharmaceutical formulation comprises at least one antioxidant compound or a pharmaceutically acceptable salt thereof and 1-O-n-Dodecyl-β-D_Maltopyranoside (DDM).

In another embodiment, the present disclosure provides a pharmaceutical formulation for treating a subject infected with a Bunyavirus such a Rift Valley fever virus, Crimean-Congo hemorrhagic fever virus or Hantavirus. The pharmaceutical formulation comprises at least one antioxidant compound or a pharmaceutically acceptable salt thereof and 1-O-n-Dodecyl-β-D_Maltopyranoside (DDM).

In another embodiment, the present disclosure provides a pharmaceutical formulation for treating a subject infected with an Arenavirus including Chapare, Lassa, Machupo and Lujo. The pharmaceutical formulation comprises at least one antioxidant compound or a pharmaceutically acceptable salt thereof and 1-O-n-Dodecyl-β-D_Maltopyranoside (DDM).

In another embodiment, the present disclosure provides a kit for treating a seizure, a neurotropic viral infection or exposure to a chemical warfare agent in a subject in need of treatment therefor. The kit comprises (a) at least one antioxidant compound or a pharmaceutically acceptable salt thereof, (b) an intranasal delivery device, and (c) an intramuscular delivery device. In some embodiments, the kit comprises a first pharmaceutical formulation in the intranasal delivery device and a second pharmaceutical formulation in the intramuscular delivery device, wherein the first and second pharmaceutical formulations both comprise one or more of said at least one antioxidant compound or a pharmaceutically acceptable salt thereof. In some embodiments, the first and second contain the same antioxidant compound in different amounts or dosages.

In another embodiment, the present disclosure provides a kit for treating a seizure, a viral infection (particularly, a neurotropic viral infection), encephalitis, exposure to a chemical warfare agent, and/or exposure to a pesticide, in a subject in need of treatment therefor. The kit comprises (a) at least one antioxidant compound or a pharmaceutically acceptable salt thereof, and (b) 1-O-n-Dodecyl-β-D_Maltopyranoside (DDM).

These and other features and advantages of the present formulations and methods will be apparent from the following detailed description, in conjunction with the appended claims.

### BRIEF DESCRIPTION OF THE FIGURES

FIG. 1 shows MR spectroscopy images obtained as described in Example 1.

FIG. 2 shows times of spectroscopic imaging in Study Period 1 of Example 1.

FIGS. 3 and 4 show results from Study Period 1 of Example 1.

FIG. 5 shows times of spectroscopic imaging in Study Period 2 of Example 1.

FIG. 6 shows results from Study Period 2 of Example 1.

FIG. 7 shows combined results from both study periods of Example 1.

FIG. 8 shows that intranasal NAC leads to a substantial increase (>50%) in brain GSH levels.

FIG. 9 shows fluctuations in GSH levels from Example 2.

### DEFINED TERMINOLOGY

It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

The terms “intranasal administration” and “intranasal delivery” mean that a pharmaceutical compound or pharmaceutically acceptable salt thereof, a biologically active fragment or variant thereof, or a formulation comprising any of the foregoing is administered to a subject through the nasal cavity or passes through nasal mucosa or epithelium or delivered to any portion of the nasal cavity.

The terms “intramuscular administration” and “intramuscular delivery” mean a pharmaceutical compound or pharmaceutically acceptable salt thereof, a biologically active fragment or variant thereof, or a formulation comprising any of the foregoing is administered to a subject by injection or deposition in muscle tissue.

The term “seizure” refers to an event when a subject experiences rapid and uncontrollable shaking, repeated and uncontrolled muscle contraction and relaxation, uncontrolled jerking movement, and/or a momentary loss of awareness. The term seizure as used herein includes convulsion and includes any seizure or convulsion event due to any physiological or environmental causes, including, but not limited to, epileptic seizures, non-epileptic seizures, vitamin B6-responsive seizures, B6-non-responsive seizures, or others. Seizure can be classified as epileptic seizures, involving a brief episode of signs and/or symptoms due to abnormal excessive or synchronous neuronal activity in the brain, and non-epileptic seizures, which are paroxysmal events that mimic an epileptic seizure but do not involve abnormal, rhythmic discharges of cortical neurons.

The term “neurotropic viral infection” means a subject is infected with a virus capable of accessing or entering the nervous system. Neurotropic viruses include alphaviruses, Dengue fever virus, Yellow fever virus, encephalitis, Japanese encephalitis virus, West Nile virus, and Zika virus of the Flaviviridae family (Volz et al 2016). These Flaviviridae viruses are also associated with oxidative stress induced neuronal death. (Yi-Ping 2019). Other examples of neurotropic viruses include Filoviruses (Filoviridae) including the Marburg virus and Ebola virus, Bunyavirales including Rift Valley fever virus, Crimean-Congo hemorrhagic fever virus, Hantavirus, Arenaviruses (Arenaviridae) including Chapare, Lassa, Machupo and Lujo. In some embodiments, the viral infection is an infection by a naturally occurring virus. In some embodiments, the viral infection is an infection by caused a genetically modified virus or a weaponized virus.

The term “chemical warfare agent” refers to is a substance whose toxic properties are intended or known to be used to kill, injure or incapacitate human beings in warfare, attack, conflict or other intentional action. Among the various types of chemical warfare agents are nerve agents (including G-agents such as sarin, cyclosarin, tabun, and soman; and V-agents such as VE, VG, VM, VR, and VX); blistering agents such as nitrogen mustard and sulfur mustard (mustard gas); asphyxiants such as carbon monoxide, chlorine, phosgene, and hydrogen sulfide gases; blood agents such as cyanide; and hydrofluoric acid.

The terms “treat”, “treating”, and “treatment” refer to a method of alleviating or abrogating a condition, disorder, or disease and/or the attendant symptoms thereof.

The term “effective amount” or “therapeutically effective amount” means a sufficient amount of the compound to treat or ameliorate a condition, disorder, or disease. When used in a medical treatment, an effective amount of one of the present compounds can be employed in pure form or, where such forms exist, in a pharmaceutically acceptable salt. Alternatively, the compound can be administered as a pharmaceutical formulation containing the compound of interest in combination with one or more pharmaceutically acceptable carriers.

The term “subject” includes humans and other primates as well as domesticated and semi-domesticated animals including, but not limited to, poultry, honeybees, cows, sheep, goats, pigs, horses, dogs, cats, rabbits, rats, mice, and the like.

The term “co-administration”, as used herein, encompasses administration of two or more agents to a subject so that both agents and/or their metabolites are present in the subject at the same time. Co-administration includes simultaneous administration in separate formulations, administration at different times in separate formulations, or administration in a formulation in which both agents are present.

The term “delivery agent” refers to a moiety capable of enhancing the delivery of a pharmaceutical drug to the central nervous system of a subject. Delivery of a pharmaceutical drug may be enhanced by various mechanisms including an increase in transport, diffusion, or stability of the pharmaceutical drug. In some embodiments, the delivery agent increases transport of a pharmaceutical compound from the nasal epithelium to the central nervous system.

As used in the specification and appended claims, and in addition to their ordinary meanings, the terms “substantial” or “substantially” mean to within acceptable limits or degree to one having ordinary skill in the art. For example, “substantially cancelled” means that one skilled in the art considers the cancellation to be acceptable.

As used in the specification and the appended claims and in addition to its ordinary meaning, the terms “approximately” and “about” mean to within an acceptable limit or amount to one having ordinary skill in the art. The term “about” generally refers to plus or minus 15% of the indicated number. For example, “about 10” may indicate a range of 8.5 to 11.5. For example, “approximately the same” means that one of ordinary skill in the art considers the items being compared to be the same.

Unless defined otherwise, all technical and scientific terms used herein have the same meaning as commonly understood by those working in the fields to which this disclosure pertain.

### DETAILED DESCRIPTION

Oxidative Stress is a Major Contributor to OP/Seizure/SE Pathophysiology and an increase in glutathione (GSH) may offset the problems caused by the oxidative stress. In an effort to increase GSH, N-Acetyl Cysteine (NAC) can be used for post injury and possibly pre-injury as a precautionary. NAC is the Limiting Substrate for GSH Synthesis and has Multiple Modes of Action.

N-Acetyl Cysteine (NAC) is a precursor to the amino acid cysteine—the rate limiting substrate in the synthesis of GSH. NAC is an antioxidant, it acts as an anti-inflammatory, leads to cysteine/glutamate exchange and affects metabotropic glutamate receptors. It has been previously shown that intranasal (IN) NAC leads to an increase in brain GSH levels in both rodents and human subjects. See Examples 1 and 2 below.

Oral NAC is readily available as a dietary supplement, and it is the active ingredient in Mucomyst, an FDA approved inhalant with a 40+ year safety history. NAC given intravenously is commonly used to treat acetaminophen overdose. The general safety of NAC has been evaluated in over 250 human clinical trials covering a wide range of medical conditions (see Šalamon et al 2019). Intranasal (IN) delivery of NAC should be more effective vs. oral due to the predicted bioavailability of IN NAC. On a battlefield setting where masks are worn by soldiers, administering IN NAC is not an option. The present disclosure provides an intramuscular (IM) injection at the time of chemical warfare exposure for those wearing protective masks, followed by intranasal delivery after a time when it's safe to remove the mask for treating both exposure to chemical warfare agents and alphavirus infections. The IN NAC medication would be 1-10×/day, although most likely 1-3×/day for 7-14 days, although it may be shorter or longer. The NAC would most likely be a 20% concentration and be readily available to deploy, pre-loaded in an IN device as well as an IM (autoinjector) device. A soldier can apply the autoinjector to himself/herself or to an injured soldier. In the case of a civilian exposure to chemical warfare, the exposed would go straight to the IN delivery. In the event of a seizure due to epilepsy, IN delivery of NAC would be used although IM delivery is another option.

The present disclosure generally relates to formulations and methods for treatment of seizures, viral infections, exposure to chemical warfare agents, exposure to pesticides and/or encephalitis. A major barrier to pharmacological treatment of brain disorders is the blood brain barrier, a network of endothelial cells coupled by tight junctions that govern solution flow and movement of compounds in and out of the brain parenchyma and that consequently reduces the effective concentration of a systemically administered compound able to reach the brain. Treatment of seizures may be enhanced using intranasal administration of pharmaceutical compounds, resulting in efficient brain delivery of the pharmaceutical compound. For example, pairing an antioxidant such as N-acetylcysteine (NAC) with intranasal administration that specifically targets the nasal epithelium may result in effective delivery of the antioxidant compound to the brain. Without wishing to be bound by any particular theory, direct nose to brain transport is thought to be possible via neural connections that travel from the central nervous system across the cribriform plate into the olfactory region of the nasal cavity. These olfactory cells pass through the nasal epithelium, which seals the nasal cavity from the central nervous system. As a result, in some embodiments, intranasal administration of pharmaceutical compounds such as antioxidant compounds produces higher therapeutic outcomes than other delivery routes.

The present disclosure provides ways to increase the robustness of treatments through rational design of improved pharmaceutical formulations or delivery methods, where others might have abandoned promising treatments. In particular, rapid and targeted delivery of antioxidant compounds following injury may arrest or diminish toxic oxidative cascades associated with seizures or neurotropic viral infections, resulting in effective treatment of those disorders.

In some embodiments, the present methods and formulations may increase levels of glutathione in a subject's brain in addition to or instead of increasing the level or concentration of a compound in either the brain or vasculature, if that compound is intranasally administered to the subject. Without wishing to be bound by any particular theory, an administered antioxidant compound may undergo conversion to cysteine in the subject's vasculature, followed by cysteine uptake across the blood brain barrier. In this manner, cysteine required for glutathione synthesis in the brain may be facilitated. In some embodiments, N-acetylcysteine is intranasally administered to a subject in order to increase a level of glutathione in the subject's brain during or after a seizure as a neurotropic viral infection.

The present disclosure also relates to treatment of a seizure, a neurotropic viral infection or exposure to a chemical warfare agent by administering a pharmaceutical agent in combination with a delivery agent to enhance transport of the pharmaceutical compound to neural tissue. It has been discovered that agents such as matrix metallopeptidase 9 (MMP-9) may be employed to enhance the delivery of pharmaceutical compounds (e.g., an antioxidant) to neural tissue. Other delivery agents such as chitosan loaded nanoparticles, lipophilic micelles, or liposomal carriers may be used to enhance delivery of a pharmaceutical compound to neural tissue as well. Accordingly, it is contemplated that any of the methods, formulations and kits discussed herein may further include matrix metallopeptidase 9 (MMP-9) or a biologically active fragment or variant thereof.

In some embodiments, the present disclosure provides a method of treating a seizure, a viral infection, encephalitis, exposure to a chemical warfare agent, and/or exposure to a pesticide. The method comprises intranasally administering to a subject an effective amount of at least one antioxidant compound or a pharmaceutically acceptable salt thereof. In some embodiments, the method also comprises administering an effective amount of a cholinergic muscarinic antagonist (for example, atropine or ipratropium), a cholinesterase reactivator (such as an oxime), or a combination thereof, to the subject. In some embodiments, the method also comprises intranasally administering a benzodiazepine.

More particularly, the method can comprise intranasally administering an effective amount of a benzodiazepine to the subject, and/or intranasally administering an effective amount of a cholinergic muscarinic antagonist, a cholinesterase reactivator, or a combination thereof to the subject. Nayzilam® and Valtoco® are products for intranasal administration of benzodiazepines. Ipratropium bromide, sold under the trade name Atrovent and others, is a cholinergic muscarinic antagonist suitable for intranasal administration. In some embodiments, the methods disclosed herein are used as adjunctive therapy with the current standard of care. For example, for methods of treating exposure to a chemical warfare agent, the present methods can comprise administering an antioxidant compound or salt thereof before, during or after administering atropine and pralidoxime, and administering diazepam or midazolam.

In some embodiments, the antioxidant compound is a small molecule. In some embodiments, the antioxidant compound is an enzyme. In some embodiments, the antioxidant compound is lipid soluble. In some embodiments, the antioxidant compound is water-soluble.

In some embodiments, the antioxidant compound is N-acetylcysteine. In some embodiments, the antioxidant compound is glutathione or a derivative thereof. In some embodiments, the antioxidant compound is coenzyme Q10. In some embodiments, the antioxidant compound is superoxide dismutase (SOD).

In some embodiments, the antioxidant compound is a pyrrolopyrimidine compound. In some embodiments, the antioxidant compound is a ubiquinone compound. In some embodiments, the antioxidant compound is a lazaroid compound.

In some embodiments, the antioxidant compound is selegiline. In some embodiments, the antioxidant compound is idebenone. In some embodiments, the antioxidant compound is probucol. In some embodiments, the antioxidant compound is tirilazad. In some embodiments, the antioxidant compound is memantine. In some embodiments, the antioxidant compound is ebselen. In some embodiments, the antioxidant compound is lipoic acid. In some embodiments, the antioxidant compound is vitamin E. In some embodiments, the antioxidant compound is vitamin C. In some embodiments, at least one antioxidant compound is a mixture of two or more of the aforementioned antioxidant compounds.

In some embodiments, the method further comprises administering a delivery agent to the subject. The delivery agent can be administered to the subject before, during or after the administration of the antioxidant compound. In some embodiments, a delivery agent is administered simultaneously with the antioxidant compound, such as when the antioxidant compound and the delivery agent are mixed in the same pharmaceutical formulation. In some embodiments, a delivery agent is administered before the administration of the antioxidant compound, for example, no more than about 15 seconds before, alternatively no more than about 30, 60, 90 or 120 seconds before, alternatively between 1 second and 10 minutes before the administration of the antioxidant compound.

In some embodiments, the method further comprises administering matrix metallopeptidase 9 (MMP-9) or a biologically active fragment or variant thereof. MMP-9 is an enzyme of the zinc-metalloproteinase family and is a member of a large family of metallopeptidases. MMP-9 is expressed at the olfactory epithelium where it plays a key role in the migration of newborn olfactory neurons. Use of matrix metallopeptidase 9 or a biologically active fragment or variant thereof can enhance the delivery of antioxidant compounds (e.g., N-acetylcysteine) to the central nervous system. Without wishing to be bound by any particular theory, it is believed that MMP-9 renders the nasal epithelium more permeable to pharmaceutical drugs by degrading type IV collagen. This degradation is believed to result in improved absorption of the pharmaceutical drug.

In some embodiments, the method further comprises administering an MMP-9 fragment or variant. In some embodiments, the MMP-9 fragment or variant is a chimeric protein comprising a biologically active fragment or variant of MMP-9.

Matrix metallopeptidase 9 may exist as various isoforms or variants. For example, the MMP-9 protein may have different lengths, sequences, or fold structures. In some embodiments, the MMP-9 comprises an MMP-9 fragment. In some embodiments, the method comprises administering at least one fragment of an MMP-9 protein. In some embodiments, the method comprises administering a fragment having a substantial sequence identity with the full-length MMP-9 protein. In some embodiments, the fragment comprises at least about 60%, at least about 75%, at least about 85%, at least about 90%, at least about 95%, at least about 96%, at least about 97%, at least about 98%, at least about 99%, at least about 99.5%, or about least about 99.9% of the sequence of full-length MMP-9 protein. However, the MMP-9 fragment may have any length consistent with the uses described herein.

In some embodiments, the MMP-9 fragment or variant thereof has at least 50% activity of the wild type version of MMP-9. Thus, in some embodiments, the MMP-9 fragment or variant thereof has a least 50%, at least 60%, at least 70%, at least 75%, at least 80%, at least 85%, at least 90%, at least 95%, at least 98%, at least 99%, or at least 99.9% activity of the wild type version of MMP-9. In some embodiments, the method further comprises administering a functionally equivalent fragment of MMP-9 (i.e., at least 75% activity of the wild type version of MMP-9). In some embodiments, the method comprises administering an MMP-9 having a full-length human MMP-9 protein.

The MMP-9 or biologically active fragment or variant thereof may be obtained from any suitable source. In some embodiments, the matrix metallopeptidase 9 or biologically active fragment or variant thereof is human-derived. In some embodiments, the matrix metallopeptidase 9 or biologically active fragment or variant thereof is murine-derived. In some embodiments, the matrix metallopeptidase 9 or biologically active fragment or variant thereof is rabbit-derived.

In some embodiments, MMP-9 or a biologically active fragment or variant thereof is administered before at least one antioxidant compound is administered to the subject. In some embodiments, MMP-9 or a fragment of variant thereof is administered simultaneously with the at least one antioxidant compound. In some embodiments, MMP-9 is administered less than about 5 minutes, less than about 10 minutes, less than about 15 minutes, less than about 20 minutes, less than about 30 minutes, less than about 1 hour, or less than about 2 hours prior to administering the at least one antioxidant compound to the subject. In some embodiments, MMP-9 is administered from about 1 minute to about 2 hours, from about 1 minutes to about 1 hour, from about 1 minute to about 30 minutes, from about 1 minute to about 20 minutes, or from about 1 minute to about 10 minutes before the at least one antioxidant is administered to the subject. In some embodiments, MMP-9 or a biologically active fragment or variant thereof is intranasally administered to the subject.

The matrix metallopeptidase 9 or a biologically active fragment or variant thereof may be used in any suitable amount. In some embodiments, MMP-9 or a biologically active fragment or variant thereof is in an amount of from about 0.001 nM to about 100 μM of a pharmaceutical formulation. Thus, in some embodiments, MMP-9 or a biologically active fragment or variant thereof is used in an amount of from about 0.001 nM to about 100 μM, from about 0.001 nM to about 50 μM, from about 0.001 nM to about 20 μM, from about 0.001 nM to about 10 μM, from about 0.001 nM to about 1 μM, from about 0.001 nM to about 500 nM, or from about 0.001 nM to about 100 nM of a pharmaceutical formulation. In some embodiments, MMP-9 or a biologically active fragment or variant thereof is used in an amount of at least about 15 μM, at least about 20 μM, at least about 25 μM, at least about 50 μM, at least about 75 μM, or at least about 100 μM of a pharmaceutical formulation.

Other delivery agents may be used in the present method. In some embodiments, delivery agents such as chitosan nanoparticles for mucoadhesion (e.g., pharmaceutical agent-loaded), micelles (e.g., lipophilic), or liposomal carriers such as archaeosomes, niosomes, novasomes, cytostomes, emulsomes, and vesosomes are used to enhance delivery of the antioxidant compound to the brain. In some embodiments, the delivery agent can be an alkylsaccharide transmucosal delivery enhancement agent (such as Intravail®) which could increase the bioavailability of NAC, an absorption-enhancing water-based gel for transmucosal and transdermal drug delivery (such as Hydrogel™) or a pectin based gelling agent (such as PecSys™) that can reduce drip and run-off.

In some embodiments, the method further comprises administering an anti-inflammatory agent to the subject. In some embodiments, the anti-inflammatory agent is a steroid. In some embodiments, the anti-inflammatory agent is a neurosteroid. In some embodiments, the anti-inflammatory agent is a lipophilic neurosteroid, a synthetic steroid, or a combination thereof. In some embodiments, the anti-inflammatory agent is progesterone.

In some embodiments, the anti-inflammatory agent is a non-steroidal compound. In some embodiments, the anti-inflammatory agent is trofinetide. In some embodiments, the anti-inflammatory agent comprises ghrelin or a variant thereof. In some embodiments, the anti-inflammatory agent is fenoprofen, ibuprofen, indomethacin, naproxen, tolmetin, or a combination thereof.

In some embodiment, the anti-inflammatory agent is administered simultaneously with the at least one antioxidant compound. In some embodiments, the anti-inflammatory agent is administered prior to administering the at least one antioxidant compound to the subject. In some embodiments, the anti-inflammatory agent is intranasally administered to the subject.

In some embodiments, the method further comprises administering acetaminophen or aspirin. In some embodiment, acetaminophen or aspirin is administered simultaneously with the at least one antioxidant compound. In some embodiments, acetaminophen or aspirin is administered prior to administering the at least one antioxidant compound to the subject.

In some embodiments, the method further comprises administering to a subject an inhibitor of a purinergic receptor. In some embodiments, the purinergic receptor is P2X4, P2X7, P2Y6, or P2Y12. In some embodiments, the inhibitor of a purinergic receptor is a thienopyridine compound. In some embodiments, the inhibitor of a purinergic receptor is clopidogrel, prasugrel, ticlopidine, ticagrelor, cangrelor, or a combination thereof. In some embodiments, the inhibitor of a purinergic receptor is administered simultaneously with the at least one antioxidant compound. In some embodiments, the inhibitor of a purinergic receptor is administered prior to administering the at least one antioxidant compound to the subject. In some embodiments, the inhibitor of a purinergic receptor is intranasally administered to the subject.

In some embodiments, the pharmaceutical compound is an antioxidant compound as described above. In some embodiments, the antioxidant compound is a pyrrolopyrimidine compound, ubiquinone compound, a lazaroid compound, or a combination thereof. In some embodiments, the pharmaceutical compound is N-acetylcysteine, glutathione, co-enzyme Q-10, superoxide dismutase, or a combination thereof. In some embodiments, the antioxidant compound is intranasally administered to the subject.

In some embodiments, the pharmaceutical compound is an anti-inflammatory agent as described above. For example, in some embodiments, the anti-inflammatory agent is a steroid or a non-steroidal compound. In some embodiments, the anti-inflammatory agent is a neurosteroid. In some embodiments, the anti-inflammatory agent is progesterone, trofinetide, ghrelin or a variant thereof, or a combination thereof. In some embodiments, the anti-inflammatory agent is fenoprofen, ibuprofen, indomethacin, naproxen, tolmetin, or a combination thereof. The anti-inflammatory agent may be administered separately or simultaneously with the at least one antioxidant compound. In some embodiments, the pharmaceutical compound is acetaminophen or aspirin.

In some embodiments, the pharmaceutical compound is an inhibitor of a purinergic receptor as described above. In some embodiments, the purinergic receptor is selected from the group consisting of P2X4, P2X7, P2Y6, and P2Y12. In some embodiments, the inhibitor of a purinergic receptor is clopidogrel, prasugrel, ticlopidine, ticagrelor, cangrelor, or a combination thereof. The inhibitor of a purinergic receptor may be administered separately or simultaneously with the pharmaceutical composition.

When the subject is treated intranasally, the compounds or formulations of the present disclosure may be administered using any suitable delivery method. In some embodiments, a solution is applied directly to the nasal epithelium or to a portion of the nasal epithelium. In some embodiments, the compound or formulation is administered via a nasal spray, nasal drops, mist, gas, or an aerosol. The spray device may be a metered spray or suspension spray device. The aerosol device may be an aerosol, metered aerosol, powder aerosol, or a spray aerosol device. In some embodiments, the compound or formulation is administered as a solution, powder, or lyophilized or a rehydrated lyophilized form.

In some embodiments, a solution (e.g., an aqueous solution) is administered to a subject using an intranasal device such as a spray bottle, metered device, or an epithelium targeting device. In some embodiments, the intranasal device propels the solution or a mist of solution into the nasal cavity using air or an inert gas. In some embodiments, a nebulizer device is used to deliver the compound or formulation to the nasal epithelium of the subject. The nebulizer device may be mechanical or electrical. The nebulizer device may be a soft mist inhaler, powder nebulizer, jet nebulizer, ultrasonic wave nebulizer, or a vibrating mesh nebulizer. In some embodiments, the device has a clock or time device, which indicates to a user or caregiver when an additional dose should be administered.

The compounds or formulations of the present disclosure can be delivered to the nasal epithelium using a variety of commercially available devices. Suitable delivery devices include VersiDoser™ delivery system, Kurve Controlled Particle Dispersion™ delivery device, Easynose® nebulizer, Impel Precision Olfactory Delivery™, ViaNase™ electronic atomizer, Aptar Pharma nasal spray pump, and Optinose breath-powered nasal delivery device. Such devices may enhance delivery of a pharmaceutical compound (e.g., antioxidant compound) to the central nervous system of a subject.

In some embodiments, the neural tissue of the brain comprises a higher concentration of antioxidant compound when administered intranasally then when administered using a different method (e.g., orally or parenterally).

In some embodiments, the antioxidant compound is administered intramuscularly to a subject. Intramuscular administration typically requires relatively lower volumes than intravenous administration. Intramuscular formulations comprising the antioxidant compound can be low volume formulations, for example 1 ml or 2 ml. In some embodiments, the antioxidant compound is administered intramuscularly at a dose up to 1 mg/kg, or up to 2 mg/kg, or up to 6 mg/kg, or up to 12 mg/kg, or more.

In some embodiments, the antioxidant compound is as described above. In some embodiments, the antioxidant compound is a small molecule. In some embodiments, the antioxidant compound is an enzyme. In some embodiments, the antioxidant compound is a pyrrolopyrimidine compound, ubiquinone compound, a lazaroid compound, or a combination thereof. In some embodiments, the pharmaceutical compound is N-acetylcysteine, glutathione, co-enzyme Q-10, superoxide dismutase, or a combination thereof. In the present disclosure, references to the “antioxidant compound” should be understood to also refer to pharmaceutically acceptable salts, hydrates, solvates, and prodrugs of antioxidant compounds.

As another aspect of the present disclosure, pharmaceutical formulations for intramuscular administration are provided. The intramuscular formulations comprise at least one antioxidant compound or a pharmaceutically acceptable salt thereof and one or more excipients. In some embodiments, at least one of the excipients is an agent for enhancing delivery of the at least one antioxidant compound or salt thereof into muscle tissue. In some embodiments, the intramuscular formulation further includes a cholinergic muscarinic antagonist such as atropine or ipratropium, and a cholinesterase reactivator such as pralidoxime chloride (2-PAM Cl) or other oxime. In some embodiments, the cholinergic muscarinic antagonist (e.g., atropine or ipratropium) is present in the intramuscular formulation at a dosage of at least 0.02 mg/kg, or in a dose amount of from 1 to 24 mg, or 2 to 12 mg, or about 6 mg. In some embodiments, the cholinesterase reactivator (e.g., 2-PAM Cl) is present in the intramuscular formulation at a dosage of from 5 mg/kg to 50 mg/kg, and/or in a dose amount of up to 4 g. In some embodiments, the intramuscular formulation comprises an antioxidant compound or salt thereof, atropine or ipratropium, a benzodiazepam and an oxime.

In some embodiments, the intramuscular formulation further comprises one or more excipients, such as of sodium chloride, sodium metabisulfite, hydrochloric acid or other acid (to adjust the formulation to a desired pH), and/or water for injection. Examples of other excipients that may be included in an intramuscular formulation include alcohols (such as ethanol), glycols (such as propylene glycol), polyols (such as glycerin), buffers (such as sodium benzoate and benzoic acid, or sodium citrate and citric acid), amino acids (such as glycine), and preservatives (such as benzyl alcohol or phenol). In some embodiments, the intramuscular formulation comprises a smell masker or other agent alleviating odor from the at least one antioxidant compound or salt thereof.

In some embodiments, the pharmaceutical formulation (including intranasal formulations and intramuscular formulations) further comprises an anti-inflammatory agent as described above. For example, in some embodiments, the anti-inflammatory agent is a steroid or a non-steroidal compound. In some embodiments, the anti-inflammatory agent is progesterone, trofinetide, ghrelin or a variant thereof, or a combination thereof. In some embodiments, the anti-inflammatory agent is fenoprofen, ibuprofen, indomethacin, naproxen, tolmetin, or a combination thereof.

In some embodiments, the pharmaceutical formulation further comprises an inhibitor of a purinergic receptor as described above. In some embodiments, the purinergic receptor is selected from the group consisting of P2X4, P2X7, P2Y6, and P2Y12. In some embodiments, the inhibitor of a purinergic receptor is clopidogrel, prasugrel, ticlopidine, ticagrelor, cangrelor, or a combination thereof.

Other delivery agents may be present in the pharmaceutical formulation. In some embodiments, delivery agents such as chitosan nanoparticles for mucoadhesion (e.g., pharmaceutical agent-loaded), micelles (e.g., lipophilic), or liposomal carriers such as archaeosomes, niosomes, novasomes, cyptosomes, emulsomes, and vesosomes are used to enhance delivery of the antioxidant compound to the brain. In some embodiments, the delivery agent can be an alkylsaccharide transmucosal delivery enhancement agent (such as Intravail®) which could increase the bioavailability of NAC, an absorption-enhancing water-based gel for transmucosal and transdermal drug delivery (such as Hydrogel™) or a pectin based gelling agent (such as PecSys™) that can reduce drip and run-off.

In some embodiments, the pharmaceutical formulation comprises one or more formulation components, including excipients that aid permeation or are suitable for use in intranasal delivery. Some examples of formulation components include pharmaceutically acceptable carriers are sugars, such as lactose, glucose and sucrose; starches such as corn starch and potato starch; cellulose and derivatives such as sodium carboxymethyl cellulose, ethyl cellulose and cellulose acetate; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; solvents such as ethyl alcohol; and buffers such as phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be included in the present formulations.

In some embodiments, the pharmaceutical formulation comprises and/or the delivery device comprises a fragrance, chemical additive, or other component that would alleviate the odor coming from NAC. Additionally, a sugar or sweetener can be added to alleviate the odor coming from NAC. Additionally, NAC can be complexed with a cyclodextrin or ion exchange resin. NAC can be incorporated into the cavity of a cyclodextrin, for instance, (2-Hydroxypropyl)-β-cyclodextrin (HP-β-CyD) also known as (2-hydroxypropyl) beta-cyclodextrin (HPBCD).

In some embodiments, the pharmaceutical formulation comprises an analgesic or flavoring compound to assist the subject's intranasal acceptance of the formulation and decrease the chances of a sneeze or other type of involuntary intranasal rejection. Suitable analgesics include but are not limited to lidocaine, pethidine, tramadol, metamizole, codeine and diclofenac. In some embodiments, the present methods and formulations further comprise an agent for enhancing delivery or and/or alleviating odor from the at least one antioxidant compound or salt thereof. For example, the agent can comprise a cyclodextrin compound in an amount effective to enhance delivery of or alleviate odor from the at least one antioxidant compound or salt thereof. An exemplary cyclodextrin compound is (2-hydroxypropyl) beta-cyclodextrin (HPBCD). In some embodiments, the agent for enhancing delivery or and/or alleviating odor is administered to the subject before administering the at least one antioxidant compound or salt thereof. Alternatively, the agent is mixed with and/or administered simultaneously with the antioxidant agent. In some embodiments, the agent is administered once in one nostril of the subject, or once in each nostril of the subject.

In some embodiments, the pharmaceutical formulation comprises an additional antioxidant to prevent NAC oxidation: adipic acid, ascorbic acid, ascorbyl palmitate, butylated hydroxyanisole, butylated hydroxytoluene, citric acid, dithiothreitol, glutamic acid, propyl gallate, sodium formaldehyde sulfoxylate, sodium metabisulfite, sodium sulfite, sodium thiosulfate, tartaric acid, thioglycerol, throurea, tocopherols, p-toluene sulfonic acid. Or a combination of hydrogen atom donor and metal chelator: citric acid, EDTA, fumaric acid and malic acid can be used to reduce NAC oxidation. In some embodiments, an intranasal delivery device holding the antioxidant compound or salt would be configured to reduce or minimize the oxidation of the antioxidant compound by storing it under an inert gas such as Argon or Nitrogen. In some embodiments, NAC solution can be kept at low pH and/or stored in packaging with low oxygen permeability such as borosilicate glass, high density polyethylene (HDPE), polypropylene (PP), polystyrene (PS), polyvinylchloride (PVC), polycarbonate (PC), unoriented polyethylene terephthalate (PET), or oriented PET. In some embodiments, the intranasal device can be unvented to prevent oxygen from entering the pre-filled container housing the therapeutic, to prevent or reduce oxidation.

In some embodiments, the pharmaceutical formulation is an aqueous solution. In some embodiments, the pharmaceutical formulation is a saline solution. In some embodiments, the pharmaceutical formulation further comprises a buffer. In some embodiments, the pharmaceutical formulation comprises phosphate buffered saline (PBS) solution.

In another embodiment, the present disclosure provides a kit for treating a subject having a seizure, a neurotropic viral infection or exposure to a chemical warfare agent. The kit comprises (a) at least one antioxidant compound or a pharmaceutically acceptable salt thereof, (b) an intranasal delivery device and (c) an intramuscular delivery device.

In another embodiment, the present disclosure provides a kit for treating a subject having a central nervous system disorder. The kit comprises (a) at least one antioxidant compound or a pharmaceutically acceptable salt thereof, and (b) matrix metallopeptidase 9 (MMP-9) or a biologically active fragment or variant thereof. In some embodiments, the kit further comprises a means for intranasally delivering (a) and (b) as described above. The matrix metallopeptidase 9 (MMP-9) or a biologically active fragment or variant thereof is as described above.

In some embodiments, the kit comprises a “pre-mixed” container comprising a formulation comprising (a) at least one antioxidant compound or a pharmaceutically acceptable salt thereof, and (b) matrix metallopeptidase 9 or a biologically active fragment or variant thereof. In some embodiments, the “pre-mixed” container is an intranasal delivery device such as a nebulizer or sprayer. In some embodiments, the “pre-mixed” container is an intramuscular injection device such as an auto-injector. In some embodiments, the kit comprises at least one prefilled intranasal delivery device and at least one prefilled intramuscular delivery device. In some embodiments, the kit comprises at least two prefilled intranasal delivery devices. In some embodiments, the kit comprises at least three prefilled intranasal delivery devices. In some embodiments, the kit comprises at least four prefilled intranasal delivery devices. In some embodiments, the kit comprises at least five prefilled intranasal delivery devices.

In some embodiments, the pharmaceutical formulation is pre-loaded into an intranasal delivery device and/or an intramuscular delivery device which is included, along with appropriate usage instructions, as part of a ready-to-use kit in a case. For example, an outer carrying case can be adapted for carrying a plurality of containers and instructions for using the components of the kit, such as by its arrangement of pockets and clips. The case can be adapted to keep the pharmaceutical formulation and/or additional chemicals stable. In some embodiments, the case is adapted to protect the pharmaceutical formulation from severe weather or conditions, i.e., rain, snow, extreme cold, extreme heat, humidity, dryness and/or to protect the pharmaceutical formulation, the intranasal delivery device and intramuscular delivery device from physical damage such as shaking or being dropped. In some embodiments, the kit or case is adapted to be stored in a soldier's backpack and be readily available in any environment or conditions.

In some embodiments, the present kits comprise one or more prefilled containers with a pharmaceutical formulation comprising at least one antioxidant compound or salt thereof. The containers can be vials, cartridges, or other containers adapted for operation with an intranasal delivery device and/or an intramuscular delivery device, such as by loading, inserting, or otherwise connecting the containers to the intranasal delivery device or the intramuscular delivery device. In some embodiments, the kit further comprises an outer carrying case for carrying the intranasal delivery device and/or the intramuscular delivery device, one or more prefilled containers, and other components. The pharmaceutical formulation is pre-formulated into a number of vials or cartridges that can be loaded into an intranasal delivery device or an intramuscular delivery device. Such vials, cartridges or other containers can be included, along with appropriate usage instructions, as part of a ready-to-use kit in an outer carrying case.

In some embodiments, the present kits comprise one or more containers with agents other than an antioxidant compound. For instance, a kit can comprise separate containers of an agent for enhancing delivery of and/or alleviating odor from an antioxidant compound. In some embodiments, the present kits comprise one or more containers that contain a fragrance or a masking agent, such as a cyclodextrin compound (e.g., (2-hydroxypropyl) beta-cyclodextrin (HPBCD)).

In some embodiments, the antioxidant compound is as described above. For example, in some embodiments, the antioxidant compound is selected from the group consisting of N-acetylcysteine, glutathione, co-enzyme Q-10, superoxide dismutase (SOD), and a combination thereof.

In some embodiments, the metallopeptidase 9 or a biologically active fragment or variant thereof is a chimeric protein or fusion protein, with the MMP-9 catalytic domain fused to a mucoadhesive peptide. In some embodiments, the matrix metallopeptidase 9 or a biologically active fragment or variant thereof comprises the full-length MMP-9 protein fused to another protein or fragment or variant thereof.

In some embodiments, the kit comprises components (a) and (b) in separate containers. Thus, in some embodiments, the kit comprises at least one antioxidant compound or a pharmaceutically acceptable salt thereof in a first container and matrix metallopeptidase 9 (MMP-9) or a biologically active fragment or variant thereof in a second container.

In some embodiments, the kit comprises instructions for using the components of the kit. In some embodiments, the kit comprises instructions for mixing the separate components, suitable conditions for the components and/or mixture, and/or suitable vessels for mixing the composition. Where a kit comprises a plurality of containers, the kit can also include instructions that direct how to use the respective containers, such as the order and timing of use. For instance, the instructions may direct the subject to administer an agent for enhancing delivery of and/or alleviating odor in one or both nostrils before the administration of an antioxidant compound, such as at a required or recommended time period before antioxidant compound administration. For example, the instructions may direct a user to administer a cyclodextrin compound such as HPBCD to one or both nostrils of a subject at least 10, 15, 30, 60 or more seconds, before administering NAC to one in both nostrils, and/or no more than 10, 5, 2 or 1 minute before administering NAC to one or both nostrils; the foregoing values can be combined to form a range. Alternatively, the instructions may direct the subject to administer an agent for enhancing delivery of and or alleviating odor and an antioxidant compound in one or both nostrils at substantially the same time as the administration of an antioxidant compound, including by mixing together before administration.

The instructions may contain how often to apply the antioxidant compound, and/or the time for administration after an initial injury, and/or the timing of application between 1 nostril to the other if the antioxidant compound is to be administered to both nostrils. In some embodiments, the instructions include the length of treatment and how often each day it should be administered. In some embodiments, the instructions may also include dosing amounts based on a subject's age, weight and/or how severe a subject's brain injury is.

In some embodiments, the kit further comprises one or more additional compounds. For example, the kit may further comprise an anti-inflammatory agent, purinergic receptor inhibitor, or another drug delivery agent such as chitosan nanoparticles for mucoadhesion (e.g., pharmaceutical agent-loaded), micelles (e.g., lipophilic), or liposomal carriers as described above. The kit may further comprise a delivery enhancing agent, a masking agent, an odor suppressing agent, a fragrance, an analgesic, or other agents. In some embodiments, the additional compounds are provided in the same or separate containers.

In some embodiments, the kit further comprises an injection device such as a syringe or an auto-injector configured for intramuscularly administering the antioxidant compound, optionally with additional therapeutic compounds. For example, the auto-injector can be filed with an antioxidant compound in combination with the current standard of care, i.e., atropine and pralidoxime followed by diazepam or midazolam. The antioxidant compound can be used in combination with any or all of those therapeutic agents or others. In some embodiments, the kit comprises a syringe or an autoinjector and 1, 2, 3 or more cartridges comprising multiple doses or multiple agents, if the drugs need to be separated before or during use.

The pharmaceutical compounds of the present disclosure (e.g., antioxidant compound) may be administered to a subject using any suitable dosage regimen. In some embodiments, the pharmaceutical compound is administered to a subject for at least 1 day. In some embodiments, the pharmaceutical compound is administered to a subject for at least 2 days. In some embodiments, the pharmaceutical compound is administered to a subject for at least 3 days. In some embodiments, the pharmaceutical compound is administered to a subject for at least 4 days. In some embodiments, the pharmaceutical compound is administered to a subject for at least 5 days. In some embodiments, the pharmaceutical compound is administered to a subject for at least 6 days. In some embodiments, the pharmaceutical compound is administered to a subject for at least 7 days. The pharmaceutical compound may be separately dosed or administered simultaneously with at least one anti-inflammatory agent, inhibitor of a purinergic receptor, or delivery agent (e.g., MMP-9 or a biologically active fragment or variant thereof) as described above.

In some embodiments, the pharmaceutical compound (e.g., antioxidant compound) is administered once daily. In some embodiments, two doses of pharmaceutical compound (e.g., antioxidant compound) are administered daily. In some embodiments, the pharmaceutical compound (e.g., antioxidant compound) is administered once daily for 1 to 30 days or for a longer period, such as 1 to 60 or 1 to 90 days, or for a period of 7, 14, 28, 56, or 84 days. In some embodiments, the pharmaceutical compound (e.g., antioxidant compound) is administered once daily for 5 to 7 days. In some embodiments, two doses of pharmaceutical compound (e.g., antioxidant compound) are administered daily for 5 to 7 days. The dosing regimen may depend on the half-life of the pharmaceutical compound (e.g., antioxidant compound). The pharmaceutical compound (e.g., antioxidant compound) may be separately dosed or administered simultaneously with any anti-inflammatory agent, inhibitor of a purinergic receptor, or delivery agent (e.g., MMP-9 or a biologically active fragment or variant thereof) as described above.

In some embodiments, at least one antioxidant compound is administered to a subject having a seizure or shortly after a seizure. Rapid and targeted delivery of the antioxidant compound during or after a seizure may arrest or reduce oxidative cascade reactions in the brain of the subject. In some embodiments, the antioxidant compound is administered to the subject during or immediately after a seizure. In some embodiments, the antioxidant compound is administered to the subject within 5 minutes, 10 minutes, 15 minutes, or 30 minutes of a seizure. In some embodiments, the antioxidant compound is administered to the subject within 1 hour of a seizure. In some embodiments, the antioxidant compound is administered to the subject within 2 hours of a seizure. In some embodiments, the antioxidant compound is administered to the subject within 4 hours of a seizure. In some embodiments, the antioxidant compound is administered to the subject within 6 hours of a seizure. In some embodiments, the antioxidant compound is administered to the subject within 12 hours of a seizure. In some embodiments, the antioxidant compound is administered to the subject within 16 hours of a seizure. In some embodiments, the antioxidant compound is administered to the subject within 24 hours of a seizure. In some embodiments, the antioxidant compound is administered to the subject within 2 days of a seizure. The antioxidant compound may be separately dosed or administered simultaneously with any anti-inflammatory agent, inhibitor of a purinergic receptor, or delivery agent (e.g., MMP-9 or a biologically active fragment or variant thereof) as described above. Intranasally administering the antioxidant compound (e.g., by a first responder), alone or in combination with at least one pharmaceutical or delivery agent, may allow for immediate treatment of a subject suffering from a seizure.

The pharmaceutical compounds (e.g., antioxidant compound) of the present disclosure can be administered in any suitable amount to treat a seizure, viral infection (particularly a neurotrophic viral infection), encephalitis, exposure to a chemical warfare agent, and/or exposure to a pesticide. In some embodiments, the total daily dose of a pharmaceutical compound (e.g., an antioxidant compound) (administered in single or divided doses) is from about 0.001 to about 100 mg/kg (i.e., mg of the compound or salt per kg body weight). Thus, in some embodiments, the total daily dose of the antioxidant compound (administered in single or divided doses) is from about 0.001 to about 100 mg/kg, from 0.001 to about 50 mg/kg, from 0.001 to about 30 mg/kg, from 0.001 to about 20 mg/kg, or from about 0.01 to about 10 mg/kg (i.e., mg of the compound or salt per kg body weight). In some embodiments, dosage unit compositions contain such amounts or submultiples thereof to make up the daily dose. In some embodiments, multiple doses per day may be used to increase the total daily dose, if desired. The amount of pharmaceutical compound may need to be adjusted based on severity, age, severity of concussion, weight, and/or size of the subject suffering from the viral infection (particularly a neurotrophic viral infection), encephalitis, exposure to a chemical warfare agent, and/or exposure to a pesticide.

In some embodiments, the brain or neural tissue of a subject comprises a higher concentration of pharmaceutical compound (e.g., antioxidant compound) or salt thereof when co-administered with MMP-9 or a biologically active fragment or variant thereof than when administered in the absence of MMP-9 or a biologically active fragment or variant thereof. In some embodiments, the cerebellum of the brain of a subject comprises a higher concentration of pharmaceutical compound (e.g., antioxidant compound) when co-administered with MMP-9 or a biologically active fragment or variant thereof than when administered in the absence of MMP-9 or a biologically active fragment or variant thereof. In some embodiments, the whole brain or a region of the brain (for example, but not limited to, the brainstem and/or the cerebral spinal fluid) of a subject comprises a higher concentration of pharmaceutical compound (e.g., antioxidant compound) when co-administered with MMP-9 or a biologically active fragment or variant thereof than when administered in the absence of MMP-9 or a biologically active fragment or variant thereof. In some embodiments, the cerebral cortex of a subject comprises a higher concentration of pharmaceutical compound (e.g., antioxidant compound) when co-administered with MMP-9 or a biologically active fragment or variant thereof than when administered in the absence of MMP-9 or a biologically active fragment or variant thereof. In some embodiments, the frontal cortex of the brain of a subject comprises a higher concentration of pharmaceutical compound (e.g., antioxidant compound) when co-administered with MMP-9 or a biologically active fragment or variant thereof than when administered in the absence of MMP-9 or a biologically active fragment or variant thereof. In some embodiments, the hippocampus of the brain of a subject comprises a higher concentration of pharmaceutical compound (e.g., antioxidant compound) when co-administered with MMP-9 or a biologically active fragment or variant thereof than when administered in the absence of MMP-9 or a biologically active fragment or variant thereof. In some embodiments, the olfactory bulb of a subject comprises a higher concentration of pharmaceutical compound (e.g., antioxidant compound) when co-administered with MMP-9 or a biologically active fragment or variant thereof than when administered in the absence of MMP-9 or a biologically active fragment or variant thereof. In some embodiments, one or more of the thalamus, the basal ganglia, the cortex, the cerebellum, the brain stem, or the nuclei, or all of those regions, of the brain of a subject comprises a higher concentration of pharmaceutical compound (e.g., antioxidant compound) when co-administered with MMP-9 or a biologically active fragment or variant thereof than when administered in the absence of MMP-9 or a biologically active fragment or variant thereof.

The methods and formulations of the present disclosure may be applied to a subject such as a human, non-human primate, or a domesticated or semi-domesticated animal. The subject may be a cow, sheep, goat, pig, horse, dog, cat, rabbit, rat, mouse, or the like. In some embodiments, the subject is a mammal. In some embodiments, the subject is a primate. In some embodiments, the subject is a human.

Pharmaceutical compounds or formulations of the present disclosure may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present disclosure may also be in the form of microparticles, microcapsules, liposomal encapsulates, and the like, as well as combinations of any two or more thereof.

The compounds of the present disclosure may be a pharmaceutically acceptable salt. Representative pharmaceutically acceptable salts include, but are not limited to, acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate, lactate, maleate, methanesulfonate, nicotinate, 2-naphthalenesulfonate, oxalate, pamoate, pectinate, persulfate, picrate, pivalate, propionate, succinate, tartrate, thiocyanate, phosphate, glutamate, bicarbonate, p-toluenesulfonate, and undecanoate.

The antioxidant compounds may be in prodrug form. Prodrugs are derivatives, which convert into the active agent when introduced into the subject in which they are used, by a chemical or biological process in vivo. Suitable prodrugs include, but are not limited to, peptide conjugates of the antioxidant compounds or MMP-9 and esters of the antioxidant compounds or MMP-9.

It is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

All patents and publications referred to herein are expressly incorporated by reference in their entireties. The citation of any publication is for its disclosure prior to the filing date and should not be construed as an admission that the present claims are not entitled to antedate such publication. Further, the dates of publication provided can be different from the actual publication dates which can be independently confirmed.

As used in the specification and appended claims, the terms “a”, “an,” and “the” include both singular and plural referents, unless the context clearly dictates otherwise. Thus, for example, “a moiety” includes one moiety and plural moieties.

### EXAMPLE

### Example 1

In this example, intranasal administration of NAC to healthy humans was studied. The small study was designed to determine the extent to which intranasal NAC leads to increased brain glutathione in healthy control subjects, as assessed using MEGA PRESS MR Spectroscopy.

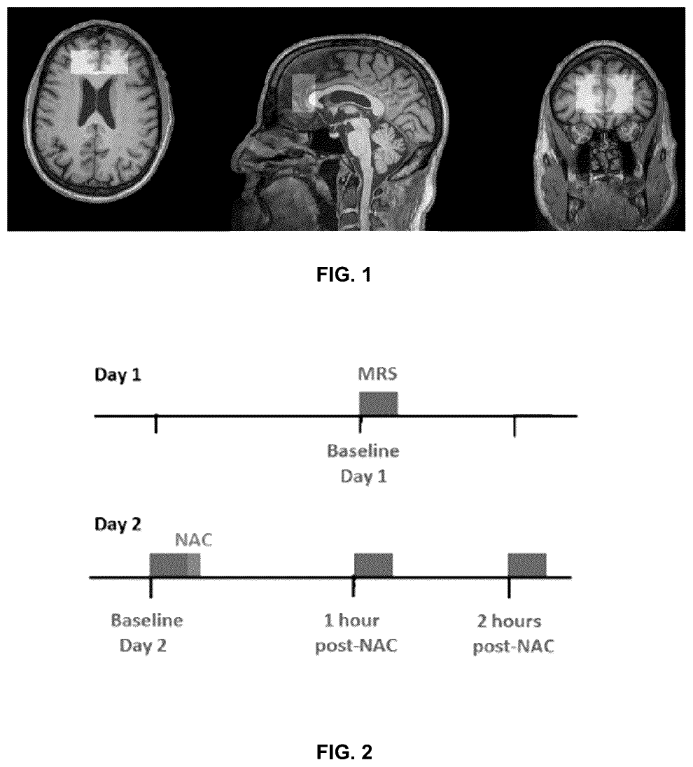

The study consisted of two study periods each involving 3 subjects and was designed to provide rapid proof-of-concept data on the ability of intranasal NAC to increase brain GSH levels. In Study Period 1, three right-handed male subjects, ages 21, 42, and 58 were evaluated. After informed consent and completion of a medical history questionnaire, each subject participated in multiple MR Spectroscopy sessions. On the first day, each subject was scanned once. A T1-weighted thin slice data set was acquired for subsequent positioning of a MR spectroscopy voxel (5×5×3 cm3) positioned with its posterior edge covering the genu of the corpus callosum, extending anteriorly into the frontal lobes. FIG. 1 shows images of the position of the MR spectroscopy voxel in the frontal lobes.

The cysteinyl β-CH2 of GSH exhibits a characteristic chemical shift at 2.95 p.p.m., which distinguishes it from other cysteine-based molecules. GSH levels were determined within the volume of interest using MEGAPRESS double-editing for the cysteinyl β-CH2 residue of GSH. Spectral editing was accomplished by refocusing GSH J-evolution during every other acquisition (ON), using a Gaussian pulse centered at the cysteinyl α-CH resonance of GSH at 4.56 p.p.m. During the alternate acquisitions (OFF), the pulse was applied symmetrically about the water peak. The difference-edited GSH spectrum was generated by subtraction of the OFF and ON spectra. Data analysis was accomplished using the GANNET software package which provided information on the GSH/Cr ratio. The Day 1 scan served as an initial baseline.

On Day 2, another baseline evaluation was performed, along with two additional scans at 1-hour and 2-hours post administration of intranasal NAC. The timing of Day 2 scans was shifted relative to Day 1, such that the time-of-day for the Day 1 baseline scan matched the time-of-day of the Day 2, 1-hour post NAC scan. This is illustrated in FIG. 2 and was done to provide control for potential circadian rhythm effects in GSH levels. In FIG. 2, green bars show times of spectroscopic imaging. The time-of-day for the baseline MRS scan on Day 1 matches the time-of-day for the 1-hour post-NAC scan on Day 2.

Intranasal NAC was delivered in the form of a 20% solution of MUCOMYST nasal spray, with 1 ml delivered to each nostril using a mucosal atomization device. Solution was delivered as six alternating bursts of 0.33 ml to each nostril over the course of 5-10 minutes.

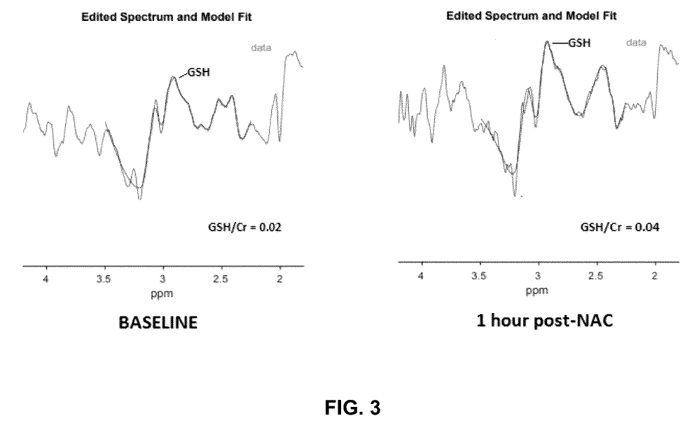

FIG. 3 shows results from Study period 1, and Table 1 provides GSH/Cr values at different time points in study period 1. FIG. 3 shows representative edited spectra for a single subject at Day 2 baseline and 1-hour post NAC scans. In looking at baseline values across subjects (shown in Table 1), there was considerable inter-individual variability, although values for a given subject were relatively stable across Day 1 and Day 2 baselines, and without evidence of an obvious circadian trend over the relatively short time-of-day window.

TABLE 1

| | Day 1, | Day 2, | Day 2, | Day 2, |
|---|---|---|---|---|
| | Baseline | Baseline | 1-hour | 2-hours |
| subject | GSH/Cr | GSH/Cr | post-NAC | post-NAC |
| 21 year old male | 0.042 | 0.038 | 0.059 | 0.052 |
| 42 year-old male | 0.017 | 0.020 | 0.037 | 0.035 |
| 58 year-old male | 0.026 | 0.024 | 0.031 | 0.048 |

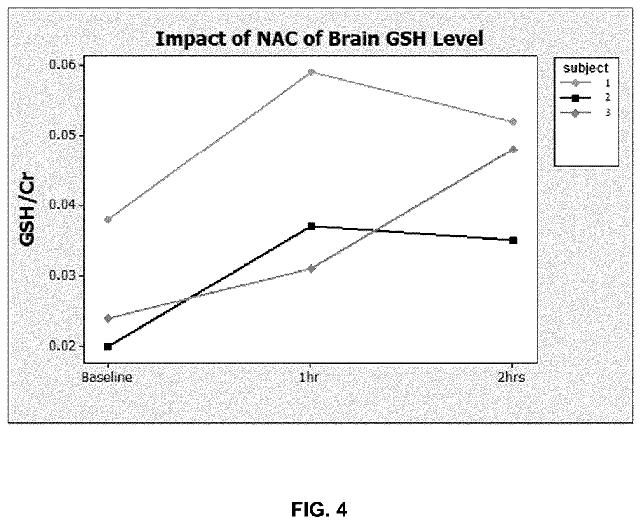

FIG. 4 illustrates the impact of Intranasal NAC on Brain GSH/Cr in the three study participants. The data demonstrates that intra-nasal NAC leads to a substantial increase in the brain GSH/Cr level that is sustained for at least 2 hours post-administration.

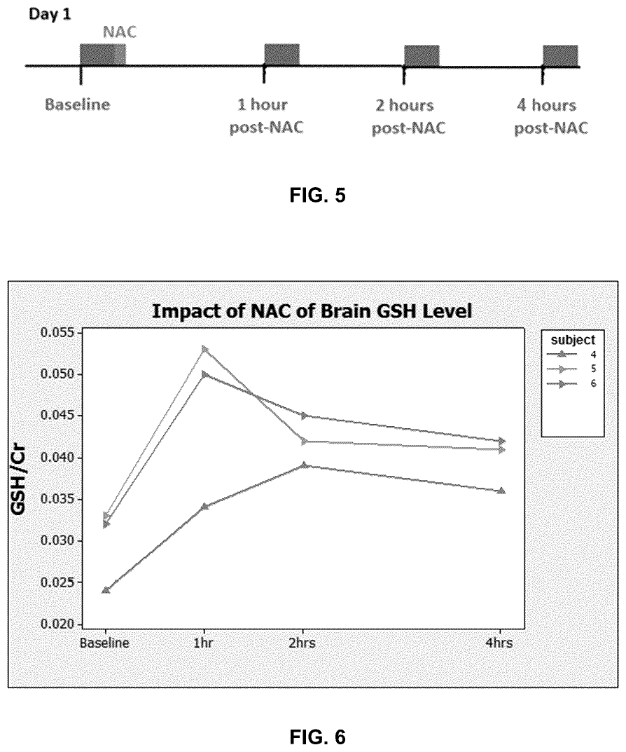

In Study Period 2, three additional right-handed male subjects, ages 23, 27, and 31 were studied. MRS data were collected on a single day, with baseline, 1 hr, 2 hrs, and 4 hrs post-NAC evaluations (as illustrated in FIG. 5). The same spectroscopic methods as used in period 1 were employed. However, a multi-dose pump intranasal delivery device was used.

FIG. 6 and Table 2 provide the main results for study period 2. FIG. 6 illustrates the impact of Intranasal NAC on Brain GSH/Cr in the three participants in Study Period 2. Table 1 shows GSH/Cr values at different time points in study period 2.

TABLE 2

| | | 1-hr | 2-hours | 4 hrs-hours |
|---|---|---|---|---|
| | Baseline | post-NAC | post-NAC | post-NAC |
| subject | GSH/Cr | GSH/Cr | GSH/Cr | GSH/Cr |
| 23 year old male | 0.024 | 0.034 | 0.039 | 0.036 |
| 27 year-old male | 0.033 | 0.053 | 0.042 | 0.041 |
| 31 year-old male | 0.032 | 0.050 | 0.045 | 0.042 |

As was seen in study period 1, intranasal administration of NAC results in a substantial increase in brain GSH/Cr levels. Importantly, the data indicate that increased levels are maintained for at least 4 hrs, with only a slow rate of decay beyond hour 2. This suggests that 2× daily dosing of intranasal NAC will be sufficient to maintain increased GSH levels.

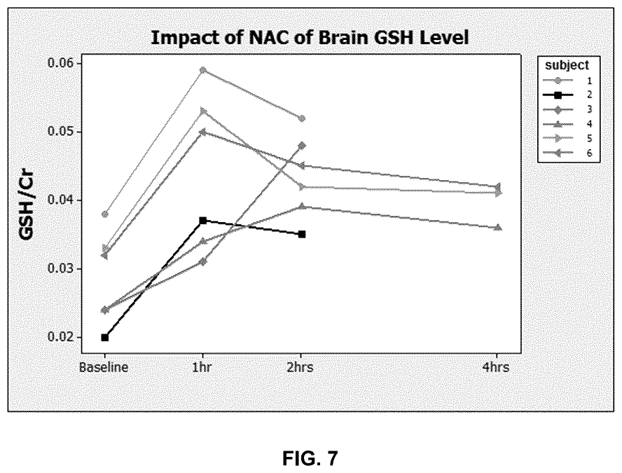

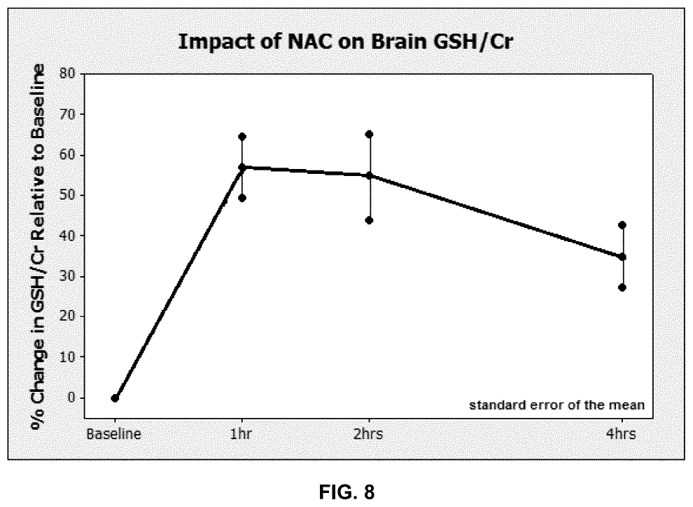

FIG. 7 shows spectroscopic data from all the individual subjects, with FIG. 8 showing the time course for the average percent change in GSH/Cr combined across all subjects. Subjects 1-3 were from study period 1 only measuring out to 2 hours, while subjects 4-6 were from study period 2, measured out to 4 hours. FIG. 8 shows that intranasal NAC leads to a substantial increase (>50%) in brain GSH levels and even at 4 hours, levels are ˜35% above baseline. Error bars are SEM. The data demonstrate a clear peak in NAC levels between 1-2 hours with sustained levels at 4 hours. At each post-NAC time point, the change in GSH/Cr level relative to baseline was statistically significant (1 hr<p. 001; 2 hours P<0.005; 4 hours p<0.05).

With respect to safety, no adverse events were reported by any subjects. No significant changes in heart-rate, breathing rate, 02 saturation or blood pressure were seen. No significant side-effects were seen although 5 of 6 subjects did voice complaints about the NAC odor, and 2 reported very mild transient nausea (<5 minutes). For the mucosal atomizer, all three subjects complained of NAC fluid into the throat. This was not found with the other nasal delivery device (the multi-dose pump).

This study provides initial evidence that intranasal NAC leads to a statistically significant increase in brain GSH as measured by MR spectroscopy.

### Example 2

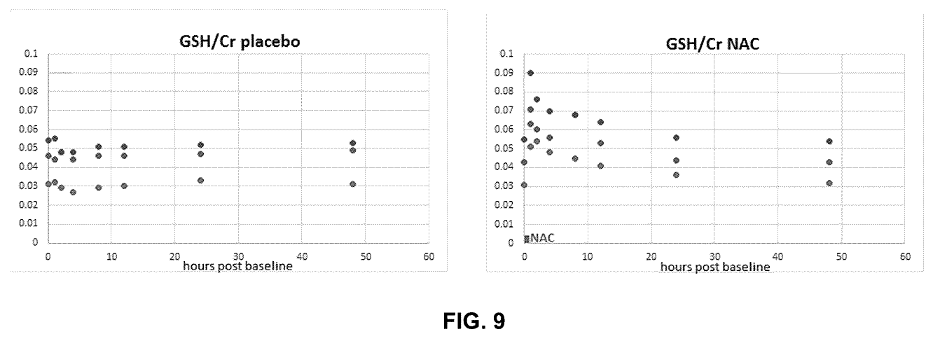

In an additional study using the same methodology as Example 1, data was collected for 3 healthy subjects showing fluctuations in GSH levels with time. Results from that study are illustrated in FIG. 9. The left panel in FIG. 9 shows the baseline levels between subjects (prior to administration of NAC), and indicates there that there is substantive variance between subject but the levels within subjects are highly stable in time in the absence of manipulation. The right panel in FIG. 9 shows levels after intranasal administration of NAC to the same subjects. Intranasal administration of NAC caused a rapid and significant increase in GSH levels, which comes down slowly with time. At 12 hours post-baseline (which is also 12 hours post-NAC administration), GSH levels were still significantly elevated following NAC, with a return to baseline at 24 hours. These data indicate that intranasal administration of NAC at 2× daily will be appropriate for maintaining increased GSH levels in the brain of the subject.

### Example 3

In this example, an PBPK analysis was performed using modeling. The analysis showed the following Table 3:

TABLE 3

| | Cmax | AUC0-10 | Tmax | BA |
|---|---|---|---|---|
| | (ng/ml) | (ng*h/ml) | (h) | (%) |
| PO NAC | 598 | 1220 | 0.67 | 9 |
| IN NAC | 770 | 1109 | 0.17 | 92 |
| IN NAC + | 1311 | 1191 | 0.03 | 98 |
| Enhancer | | | | |

The PO data was measured by taking blood samples. The IN data was based on a predictive model. Not only is the predicted bioavailability greater, but the time to maximum concentration (Tmax) is decreased by about 4 fold. Additional benefits of IN compared to other methods of delivery include that IN is easy to administer, can be applied on an unconscious person or someone not capable of swallowing a pill (such as during a seizure), has reduced or no side effects and can be administered outside of a hospital or clinic.

### EXEMPLARY EMBODIMENTS

Exemplary embodiments provided in accordance with the presently disclosed subject matter include, but are not limited to, the claims and the following embodiments:

A. A method of treating a seizure, viral infection, encephalitis, exposure to a chemical warfare agent, and/or exposure to a pesticide, the method comprising intranasally administering to a subject in need of treatment for a seizure, viral infection, encephalitis, exposure to a chemical warfare agent, and/or exposure to a pesticide, an effective amount of at least one antioxidant compound or a pharmaceutically acceptable salt thereof.

A1. The method of embodiment A, wherein the subject is in need of treatment for a seizure.

A2. The method of embodiment A, wherein the subject is in need of treatment for a viral infection.

A3. The method of embodiment A, wherein the subject is in need of treatment for exposure to a chemical warfare agent.

A4. The method of embodiment A, wherein the subject is in need of treatment for exposure to a pesticide, such as an organophosphate.

A5. The method of embodiment A, wherein the subject is in need of treatment for encephalitis, such as encephalitis caused by viral infection, exposure to a chemical warfare agent, and/or exposure to a pesticide.

A6. The method of any of embodiments A to A5, wherein the antioxidant compound is selected from the group consisting of N-acetylcysteine (NAC), glutathione, co-enzyme Q-10, superoxide dismutase (SOD), and a combination thereof.

A7. The method of any of embodiments A to A6, wherein the method further comprises administering to the subject matrix metallopeptidase 9 (MMP-9) or a biologically active fragment or variant thereof.

A8. The method of embodiment A7, wherein the matrix metallopeptidase 9 (MMP-9) or a biologically active fragment or variant thereof is intranasally administered to the subject prior to administering the at least one antioxidant compound.

A9. The method of any of embodiments A to A8, wherein the method further comprises administering to the subject trofinetide, progesterone, neurosteroid, a ghrelin compound, a salt thereof, or a combination thereof.

A10. The method of any of embodiments A to A9, wherein the method further comprises administering to the subject an inhibitor of a purinergic receptor selected from the group consisting of P2X4, P2X7, P2Y6, and P2Y12.

A11. The method of any of embodiments A to A10, wherein the method further comprises administering to the subject a cholinergic muscarinic antagonist, a cholinesterase reactivator, or both.

A12. The method of embodiment A11, wherein the cholinergic muscarinic antagonist is atropine, ipratropium, or a pharmaceutically acceptable salt thereof; and the cholinesterase reactivator is pralidoxime chloride (2-PAM Cl) or other oxime.

A13. The method of embodiment A11 or A12, further comprising administering a benzodiazepam to the subject.

A14. The method of embodiments A or A3, wherein the subject is exposed to a chemical warfare agent selected from the group consisting of nerve agents (including organophosphates; G-agents such as sarin, cyclosarin, tabun, and soman; and V-agents such as VE, VG, VM, VR, and VX); blistering agents such as nitrogen mustard, sulfur mustard or other mustard gas; asphyxiants such as carbon monoxide, chlorine, phosgene, and hydrogen sulfide gases; blood agents such as cyanide; and hydrofluoric acid.

A15. The method of embodiments A or A2, wherein the subject is in need of treatment for infection with a neurotropic virus.

A16. The method of embodiments A or A2, wherein the subject is in need of treatment for infection with an alphavirus.

A17. The method of any of embodiments A, A2, A15 or A16, wherein the virus is a naturally occurring virus.

A18. The method of any of embodiments A, A2, A15 or A16, wherein the virus is a genetically modified virus or a weaponized virus.

A19. The method of any of embodiments A to A18, wherein the at least one antioxidant is administered in combination with a chitosan loaded nanoparticle, a lipophilic micelle, or a liposomal carrier.

A20. The method of any of embodiments A to A19, wherein the subject is a human.

A21. The method of any of embodiments A to A20 wherein the method comprises administering a total daily dose of the antioxidant compound or salt thereof from about 0.001 to about 100 mg/kg

B. A pharmaceutical formulation for treatment of a seizure, viral infection, encephalitis, exposure to a chemical warfare agent, and/or exposure to a pesticide comprising at least one antioxidant compound or a pharmaceutically acceptable salt thereof and an excipient.

B1. The pharmaceutical formulation of embodiment B, wherein the pharmaceutical formulation is for treatment of a seizure.

B2. The pharmaceutical formulation of embodiment B, wherein the pharmaceutical formulation is for treatment of a viral infection, such as a neurotropic viral infection or an alphavirus infection, including naturally occurring viruses and genetically modified or weaponized viruses.

B3. The pharmaceutical formulation of embodiment B, wherein the pharmaceutical formulation is for treatment of exposure to a chemical warfare agent.

B4. The pharmaceutical formulation of embodiment B, wherein the pharmaceutical formulation is for treatment of exposure to a pesticide.

B5. The pharmaceutical formulation of embodiment B, wherein the pharmaceutical formulation is for treatment of encephalitis.

B6. The pharmaceutical formulation of any of embodiments B to B5, wherein the antioxidant compound is selected from the group consisting of N-acetylcysteine (NAC), glutathione, co-enzyme Q-10, superoxide dismutase (SOD), and a combination thereof.

B7. The pharmaceutical formulation of any of embodiments B to B6, wherein the pharmaceutical formulation further comprising a matrix metallopeptidase 9 (MMP-9) or a biologically active fragment or variant thereof wherein the MMP-9 or a biologically active fragment or variant thereof is a chimeric protein with the MMP-9 catalytic domain fused to a mucoadhesive peptide.

B8. The pharmaceutical formulation of any of embodiments B to B7, wherein the pharmaceutical formulation further comprises trofinetide, progesterone, a neurosteroid, a ghrelin compound, or a combination thereof.

B9. The pharmaceutical formulation of any of embodiments B to B8, wherein the pharmaceutical composition further comprises an inhibitor of a purinergic receptor selected from the group consisting of P2X4, P2X7, P2Y6, and P2Y12.

B10. The pharmaceutical formulation of any of embodiments B to B9, wherein the pharmaceutical composition further comprises a cholinergic muscarinic antagonist, a cholinesterase reactivator, or both.

B11. The pharmaceutical formulation of any of embodiments B to B9, wherein the pharmaceutical composition further comprises atropine, ipratropium, or a pharmaceutically acceptable salt thereof.

B12. The pharmaceutical formulation of any of embodiments B to B9 or B11, wherein the pharmaceutical composition further comprises pralidoxime chloride (2-PAM Cl).

B13. The pharmaceutical formulation of any of embodiments B to B9 or B11, wherein the pharmaceutical composition further comprises an oxime.

B14. The pharmaceutical formulation of any of embodiments B to B13, wherein the pharmaceutical composition further comprises a benzodiazepam.

C. A kit for treating a seizure, viral infection (particularly a neurotrophic viral infection), encephalitis, exposure to a chemical warfare agent, and/or exposure to a pesticide. The kit comprises (a) at least one antioxidant compound or a pharmaceutically acceptable salt thereof, and (b) a delivery device containing the at least one antioxidant compound or salt thereof.

C1. The kit of embodiment C, wherein the antioxidant compound is N-acetylcysteine (NAC).

C2. The kit of embodiment C or C1, wherein the delivery device comprises an intranasal delivery device.

C3. The kit of embodiment C2, wherein the intranasal delivery device is a sprayer or nebulizer.

C4. The kit of any embodiments C to C3, wherein the delivery device comprises an intramuscular delivery device.

D. A method of treating exposure to a chemical warfare agent and/or exposure to a pesticide, the method comprising intranasally administering to a human subject in need of treatment for exposure to a chemical warfare agent and/or exposure to a pesticide, an effective amount of at least one antioxidant compound or a pharmaceutically acceptable salt thereof.

D1. The method of embodiment D, wherein the subject has a protective facial covering, and the method comprises intramuscularly administering the at least one antioxidant compound or salt thereof without removing the protective facial covering.

D2. The method of embodiment D or D1, further comprising detecting presence or level of the chemical warfare agent and/or the pesticide in an environment around the subject.

D3. The method of embodiment D3, wherein if the chemical warfare agent and/or the pesticide is not detected or is below a threshold, the method further comprises removing the protective facial covering; and intranasally administering the at least one antioxidant compound or salt thereof to the subject.

D4. The method of embodiment D3, wherein if the chemical warfare agent and/or the pesticide is detected at a level above the threshold, the method further comprises refraining from removing the protective facial covering; and intramuscularly administering a second dose of the at least one antioxidant compound or salt thereof.

D5. The method of any of embodiment D to D4, wherein the antioxidant compound is selected from the group consisting of N-acetylcysteine (NAC), glutathione, co-enzyme Q-10, superoxide dismutase (SOD), and a combination thereof.

D6. The method of any of embodiments D to D5, wherein the method further comprises administering to the subject a cholinergic muscarinic antagonist, a cholinesterase reactivator, or both.

D7. The method of embodiment D6, wherein the cholinergic muscarinic antagonist is atropine or ipratropium, and the cholinesterase reactivator is pralidoxime chloride (2-PAM Cl) or other oxime.

D8. The method of any of embodiments D to D7, further comprising administering a benzodiazepam to the subject.

D9. The method of any of embodiments D to D8, wherein the method further comprises administering to the subject matrix metallopeptidase 9 (MMP-9) or a biologically active fragment or variant thereof.

D10. The method of any of embodiments D to D9, wherein the method further comprises administering to the subject a non-steroid anti-inflammatory agent.

D11. The method of any of embodiments D to D10, wherein the method further comprises administering to the subject trofinetide, progesterone, neurosteroid, a ghrelin compound, a salt thereof, or a combination thereof.

D12. The method of any of embodiments D to D11, wherein the method further comprises administering to the subject an agent for enhancing delivery of and/or alleviating odor from the at least one antioxidant compound or salt thereof.

D13. The method of embodiment D12, wherein the agent comprises a cyclodextrin compound in an amount effective to enhance delivery of and/or alleviate odor from the at least one antioxidant compound or salt thereof.

D14. The method of embodiment D12, wherein the agent comprises (2-hydroxypropyl) beta-cyclodextrin (HPBCD), and the agent is administered to the subject before administering the at least one antioxidant compound or salt thereof.

D15. The method of any of embodiments D to D14, wherein the at least one antioxidant compound or salt thereof is administered as a pharmaceutical formulation comprising the at least one antioxidant in combination with one or more sweetening, flavoring or perfuming agents.

D16. The method of any of embodiments D to D15, wherein the at least one antioxidant compound or salt thereof is administered from 1 to 30 days.

E. A pharmaceutical formulation for intramuscular administration comprising at least one antioxidant compound or a pharmaceutically acceptable salt thereof and one or more excipients.

E1. The pharmaceutical formulation of embodiment E, wherein at least one of said one or more excipients is an agent for enhancing delivery of the at least one antioxidant compound or salt thereof into muscle tissue.

E2. The pharmaceutical formulation of embodiments E or E1, further comprising a cholinergic muscarinic antagonist, a cholinesterase reactivator, or both.

E3. The pharmaceutical formulation of any of embodiments E to E2, wherein the cholinergic muscarinic antagonist is atropine, ipratropium, or a pharmaceutically acceptable salt thereof.

E4. The pharmaceutical formulation of any of embodiments E to E3, wherein the cholinesterase reactivator is pralidoxime chloride (2-PAM Cl) or other oxime.

E5. The pharmaceutical formulation of any of embodiments E or E1, wherein the formulation comprises atropine, a benzodiazepam and an oxime.

E6. The pharmaceutical formulation of any of embodiments E to E5, wherein said one or more excipients comprises an agent for alleviating odor from the at least one antioxidant compound or salt thereof.

E7. The pharmaceutical formulation of any of embodiments E to E6, wherein the formulation comprises a cyclodextrin compound in an amount effective to enhance delivery of and or alleviate odor from the at least one antioxidant compound or salt thereof.

E8. The pharmaceutical formulation of any of embodiments E to E7, wherein the formulation comprises (2-hydroxypropyl) beta-cyclodextrin (HPBCD).

E9. The pharmaceutical formulation of any of embodiments E to E8, further comprising an analgesic.

E10. The pharmaceutical formulation of any of embodiments E to E9, further comprising trofinetide, progesterone, neurosteroid, a ghrelin compound, a salt thereof, or a combination thereof.

E11. The pharmaceutical formulation of any of embodiments E to E10, further comprising further comprises au inhibitor of a purinergic receptor selected from the group consisting of P2X4, P2X7, P2Y6, and P2Y12.

E12. The pharmaceutical formulation of any of embodiments E to E11, further comprising one or more additives that prevents or slows oxidation of the at least one antioxidant compound or salt thereof.

E14. The pharmaceutical formulation of any of embodiments E to E13, wherein the antioxidant compound is selected from the group consisting of N-acetylcysteine (NAC), glutathione, co-enzyme Q-10, superoxide dismutase (SOD), and a combination thereof.

F. A kit for treating a subject having a seizure, encephalitis, a viral infection, exposure to a chemical warfare agent, and/or exposure to a pesticide, the kit comprising: (a) at least one antioxidant compound or a pharmaceutically acceptable salt thereof, (b) an intranasal delivery device and (c) an intramuscular delivery device.

F1. The kit of embodiment F, wherein the kit comprises at least one prefilled intranasal delivery device and/or at least one prefilled intramuscular injection device.

F2. The kit of embodiment F or F1, wherein the kit further comprises instructions for using the components of the kit.

F3. The kit of any of embodiments F to F2, wherein the at least one antioxidant compound or salt thereof is contained in one or more pharmaceutical formulations.

F4. The kit of embodiment F3, wherein a first of the one or more pharmaceutical formulations is prefilled into the intranasal delivery device, and a second of the one or more pharmaceutical formulations is prefilled in the intramuscular delivery device; wherein the kit further comprises instructions for using the intranasal delivery device and the intramuscular delivery device.

F5. The kit of any of embodiments F to F4, wherein the intramuscular delivery device comprises from 1 mg/ml to 10 mg/ml of the at least one antioxidant compound or salt thereof.

F6. The kit of any of embodiments F to F5, wherein the intramuscular delivery device comprises from 1 mg to 20 mg of the at least one antioxidant compound or salt thereof.

F7. The kit of embodiment F5 or F6, where the at least one antioxidant compound or salt thereof is in a volume of 2 ml, for example 10 mg/2 ml or 5 mg/2 ml.

F8. The kit of any of embodiments F to F7, wherein the intramuscular delivery device is an autoinjector.

F9. The kit of any of embodiments F to F8, wherein the intramuscular delivery device contains a gas (such as nitrogen), argon, or other inert gas overlaying the at least one antioxidant compound or salt thereof.

F10. The kit of any of embodiments F to F9, wherein the intranasal delivery device is an unvented nasal pump. It is contemplated that an unvented pump will reduce or prevent the ingress of oxygen into the vial holding NAC after each spray, thereby preventing, reducing of delaying the oxidation of NAC or other agent.

F11. The kit of any of embodiments F to F10, wherein the at least one antioxidant compound or salt thereof is contained in a pharmaceutical formulation, and the pharmaceutical formulation is prefilled into a plurality of containers adapted for connection to the intranasal delivery device and the intramuscular delivery device; wherein the kit further comprises instructions using the components of the kit.

F12. The kit of any embodiments F to F11, wherein the kit further comprises a sensor for detecting a chemical warfare agent and/or a pesticide.

F13. The kit of any of embodiments F to F12, wherein the kit further comprises an outer carrying case for carrying the intranasal delivery device and the intramuscular delivery device.

F14. The kit of embodiment F13, wherein the outer carrying case is adapted for carrying a plurality of containers and instructions for using the components of the kit.

F15. The kit of any of embodiments F to F14, wherein the kit further comprises a non-steroidal anti-inflammatory agent.

F16. The kit of any of embodiments F to F15, wherein the kit further comprises an agent for enhancing delivery of and/or alleviating odor from the at least one antioxidant compound or salt thereof, wherein the agent for enhancing delivery of and/or alleviating odor and the at least one antioxidant compound or salt thereof are in separate containers in the kit.

F17. The kit of any of embodiments F to F16, wherein the at least one antioxidant compound or salt thereof is provided in a pharmaceutical formulation according to any of embodiments B to B14 or any of embodiments E to E14.

### REFERENCES

McDonough J H Jr, Shih T M. Neuropharmacological mechanisms of nerve agent-induced seizure and neuropathology. Neurosci Biobehav Rev. 1997 September; 21(5):559-79.
Joosen M J, Smit A B, van Helden H P. Treatment efficacy in a soman-poisoned guinea pig model: added value of physostigmine?. Arch Toxicol. 2011; 85(3):227-237.
McDonough J H Jr, Clark T R, Slone T W Jr, Zoeffel D, Brown K, Kim S, Smith C D. Neural lesions in the rat and their relationship to EEG delta activity following seizures induced by the nerve agent soman. Neurotoxicology. 1998 June; 19(3):381-91.
Miyaki, K., Nishiwaki, Y., Maekawa, K., Ogawa, Y., Asukai, N., Yoshimura, K., Etoh, N., Matsumoto, Y., Kikuchi, Y., Kumagai, N., & Omae, K. (2005). Effects of sarin on the nervous system of subway workers seven years after the Tokyo subway sarin attack. Journal of occupational health, 47(4), 299-304.
Yanagisawa N, Morita H, Nakajima T. Sarin experiences in Japan: acute toxicity and long-term effects. J Neurol Sci. 2006 Nov. 1; 249(1):76-85. doi: 10.1016/j.jns.2006.06.007. Epub 2006 Sep. 7.
Steenland K, Jenkins B, Ames R G, O'Malley M, Chrislip D, Russo J. Chronic neurological sequelae to organophosphate pesticide poisoning. Am J Public Health. 1994 May; 84(5):731-6.
McDonough J H Jr, Shih T M. Neuropharmacological mechanisms of nerve agent-induced seizure and neuropathology. Neurosci Biobehav Rev. 1997 September; 21(5):559-79.
Raveh L, Brandeis R, Gilat E, Cohen G, Alkalay D, Rabinovitz I, et al. Anticholinergic and antiglutamatergic agents protect against soman-induced brain damage and cognitive dysfunction. Toxicol Sci 2003; 75:108-16.
Weissman B A, Raveh L. Therapy against organophosphate poisoning: the importance of anticholinergic drugs with antiglutamatergic properties. Toxicol Appl Pharmacol. 2008 Oct. 15; 232(2):351-8.
Gilat E, Kadar T, Levy A, Rabinovitz I, Cohen G, Kapon Y, Sahar R, Brandeis R. Anticonvulsant treatment of sarin-induced seizures with nasal midazolam: an electrographic, behavioral, and histological study in freely moving rats. Toxicol Appl Pharmacol. 2005; 209:74-85.
Lewine J D, Weber W, Gigliotti A, McDonald J D, Doyle-Eisele M, Bangera N, Paulson K, Magcalas C, Hamilton D A, Garcia E, Raulli R, Laney J. Addition of ketamine to standard-of-care countermeasures for acute organophosphate poisoning improves neurobiological outcomes. Neurotoxicology. 2018 December; 69:37-46.
Vezzani A, Balosso S, Ravizza T. Neuroinflammatory pathways as treatment targets and biomarkers in epilepsy. Nat Rev Neurol. 2019 August; 15(8):459-472. doi: 10.1038/s41582-019-0217-x. Epub 2019 Jul. 1. PMID: 31263255.
Vezzani A, Sperk G, Colmers W F. Neuropeptide Y: emerging evidence for a functional role in seizure modulation. Trends Neurosci. 1999 January; 22(1):25-30.
Viviani B, Bartesaghi S, Gardoni F, Vezzani A, Behrens M M, Bartfai T, Binaglia M, Corsini E, Di Luca M, Galli C L, Marinovich M. Interleukin-1beta enhances NMDA receptor-mediated intracellular calcium increase through activation of the Src family of kinases. J Neurosci. 2003 Sep. 24; 23(25):8692-700.
Balosso S, Maroso M, Sanchez-Alavez M, Ravizza T, Frasca A, Bartfai T, Vezzani A. A novel non-transcriptional pathway mediates the proconvulsive effects of interleukin-1beta. Brain. 2008 December; 131 (Pt 12):3256-65.
Sayyah M, Javad-Pour M, Ghazi-Khansari M. The bacterial endotoxin lipopolysaccharide enhances seizure susceptibility in mice: involvement of proinflammatory factors: nitric oxide and prostaglandins. Neuroscience. 2003; 122(4):1073-80.
Galic M A, Riazi K, Heida J G, et al. Postnatal inflammation increases seizure susceptibility in adult rats. J Neurosci. 2008; 28(27):6904-6913.
Scaffidi P., Misteli T., Bianchi M. E. (2002). Release of chromatin protein HMGB1 by necrotic cells triggers inflammation. Nature 418, 191-195.
Ferenc Müller, Csaba Ádori, Miklós Sass (2004). Autophagic and apoptotic features during programmed cell death in the fat body of the tobacco hornworm (Manduca sexta) European Journal of Cell Biology Volume 83, Issue 2, Pages 67-78.
Balosso S, Liu J, Bianchi M E, Vezzani A. Disulfide-containing high mobility group box-1 promotes N-methyl-D-aspartate receptor function and excitotoxicity by activating Toll-like receptor 4-dependent signaling in hippocampal neurons. Antioxid Redox Signal. 2014 Oct. 20; 21(12):1726-40.
Maroso M, Balosso S, Ravizza T, Liu J, Aronica E, Iyer A M, Rossetti C, Molteni M, Casalgrandi M, Manfredi A A, Bianchi M E, Vezzani A. Toll-like receptor 4 and high-mobility group box-1 are involved in ictogenesis and can be targeted to reduce seizures. Nat Med. 2010 April; 16(4):413-9.
King G L. The role of inflammatory cytokines in diabetes and its complications. J Periodontol. 2008 August; 79 (8 Suppl): 1527-34.
Singh K, Ritchey J W, Confer A W. Mannheimia haemolytica: bacterial-host interactions in bovine pneumonia. Vet Pathol. 2011 March; 48(2):338-48.
Vezzani, A., Fujinami, R. S., White, H. S. et al. Infections, inflammation and epilepsy. Acta Neuropathol 131, 211-234 (2016).
Chen C, Zhang X R, Ju Z Y, He W F. [Advances in the research of mechanism and related immunotherapy on the cytokine storm induced by coronavirus disease 2019]. Zhonghua Shao Shang Za Zhi. 2020 Jun. 20; 36(6):471-475.
DePaula-Silva, A. B., Sonderegger, F. L., Libbey, J. E. et al. The immune response to picornavirus infection and the effect of immune manipulation on acute seizures. J. Neurovirol. 24, 464-477 (2018).
Ronca S E, Dineley K T, Paessler S. Neurological Sequelae Resulting from Encephalitic Alphavirus Infection. Front Microbiol. 2016 Jun. 20; 7:959.
Volz A, Lim S, Kaserer M, Lülf A, Marr L, Jany S, Deeg C A, Pijlman G P, Koraka P, Osterhaus A D, Martina B E, Sutter G. Immunogenicity and protective efficacy of recombinant Modified Vaccinia virus Ankara candidate vaccines delivering West Nile virus envelope antigens. Vaccine. 2016 Apr. 7; 34(16):1915-26.
Zhang Z, Rong L, Li Y P. Flaviviridae Viruses and Oxidative Stress: Implications for Viral Pathogenesis. Oxid Med Cell Longev. 2019 Aug. 19; 2019:1409582.
Geraci M J. Mustard gas: Imminent danger or eminent threat? Ann Pharmacother. 2008; 42(2):237-246
Weinberger B, Malaviya R, Sunil V R, Venosa A, Heck D E, Laskin J D, Laskin D L. Mustard vesicant-induced lung injury: Advances in therapy. Toxicol Appl Pharmacol. 2016 Aug. 15; 305:1-11
Šalamon Š, Kramar B, Marolt T P, Poljšak B, Milisav I. Medical and Dietary Uses ofN-Acetylcysteine. Antioxidants (Basel). 2019 Apr. 28; 8(5):111.

1. A method of treating a seizure, viral infection, encephalitis, exposure to a chemical warfare agent, and/or exposure to a pesticide, wherein the method comprises:
intranasally administering to a subject in need of treatment for a seizure, viral infection, encephalitis, exposure to a chemical warfare agent, and/or exposure to a pesticide, an effective amount of at least one antioxidant compound or a pharmaceutically acceptable salt thereof.

2. The method of claim 1, wherein the subject is in need of treatment for a seizure.

3. The method of claim 1, wherein the subject is in need of treatment for a viral infection.

4. The method of claim 1, wherein the subject is in need of treatment for exposure to a chemical warfare agent.

5. The method of claim 1, wherein the subject is in need of treatment for exposure to a pesticide.

6. The method of claim 5, wherein the subject is in need of treatment for exposure to an organophosphate.

7. The method of claim 1, wherein the subject is in need of treatment for encephalitis, such as encephalitis caused by viral infection, exposure to a chemical warfare agent, and/or exposure to a pesticide.

8. The method of claim 1, wherein the antioxidant compound is selected from the group consisting of N-acetylcysteine (NAC), glutathione, co-enzyme Q-10, superoxide dismutase (SOD), and a combination thereof.

9. The method of claim 1, wherein the antioxidant compound is N-acetylcysteine (NAC) or a pharmaceutically acceptable salt thereof.

10. The method of claim 1 or claim 9, wherein the method further comprises administering to the subject an inhibitor of a purinergic receptor selected from the group consisting of P2X4, P2X7, P2Y6, and P2Y12.

11. The method of claim 10, wherein the method further comprises administering to the subject a cholinergic muscarinic antagonist, a cholinesterase reactivator, or both.

12. The method of claim 11, wherein the cholinergic muscarinic antagonist is atropine, ipratropium, or a pharmaceutically acceptable salt thereof; and the cholinesterase reactivator is pralidoxime chloride (2-PAM Cl) or other oxime.

13. The method of claim 10, further comprising administering a benzodiazepam to the subject.

14. The method of claim 1, wherein the subject is in need of treatment for infection with a neurotropic virus.

15. The method of claim 1, wherein the subject is in need of treatment for infection with an alphavirus.

16. The method of claim 14 or claim 15, wherein the virus is a naturally occurring virus.

17. The method of claim 14 or claim 15, wherein the virus is a genetically modified virus or a weaponized virus.

18. The method of claim 1, wherein the at least one antioxidant is administered in combination with a chitosan loaded nanoparticle, a lipophilic micelle, or a liposomal carrier.

19.-86. (canceled)

87. The method of claim 1, wherein the at least one antioxidant is administered in combination with one or more of adipic acid, ascorbic acid, ascorbyl palmitate, butylated hydroxyanisole, butylated hydroxytoluene, citric acid, dithiothreitol, glutamic acid, propyl gallate, sodium formaldehyde sulfoxylate, sodium metabisulfite, sodium sulfite, sodium thiosulfate, tartaric acid, thioglycerol, throurea, tocopherols, p-toluene sulfonic acid, EDTA, fumaric acid or malic acid.

88. The method of claim 1, wherein the at least one antioxidant is administered in combination with 1-O-n-Dodecyl-β-D_Maltopyranoside (DDM).
